(19) United States
(12) Patent Application Publication
(10) Pub. No.: US 2021/0340502 A1
(43) Pub. Date: Nov. 4, 2021

(54) Generation of Post-Mitotic Migratory Cortical Interneurons

(71) Applicants: The McLean Hospital Corporation, Belmont, MA (US); New York Medical College, Valhalla, NY (US)

(72) Inventor: Sangmi Chung, Ardsley, NY (US)

(21) Appl. No.: 17/280,225

(22) PCT Filed: Sep. 26, 2019
(86) PCT No.: PCT/US2019/053089

§ 371 Date: Mar. 26, 2021

Related U.S. Application Data
(60) Provisional application No. 62/738,492, filed on Sep. 28, 2018.

(51) Int. Cl. C12N 5/074; A01N 1/02

(52) U.S. Cl. CPC C12N 5/0696; C12N 2500/34; A01N 1/0221

(57) ABSTRACT

The present disclosure provides populations of synchronized post-mitotic migratory cortical interneurons (cINS) derived from pluripotent stem cells and cell culture methods for generating said populations of cINs. The disclosure also provides efficient methods for cryopreservation of the derived cINs.

### CROSS-REFERENCE TO RELATED APPLICATION

This application claims the benefit of and priority to U.S. Provisional Patent Application No. 62/738,492, filed on Sep. 28, 2018, which is incorporated herein by reference in its entirety.

The present disclosure provides populations of synchronized post-mitotic migratory cortical interneurons (cINS) derived from pluripotent stem cells and cell culture methods for generating said populations of cINs. The disclosure also provides efficient methods for cryopreservation of the derived cINs.

### BACKGROUND OF THE INVENTION

Cortical interneurons (cINs), especially those subtypes that express PV and SST, are derived from the MGE in subpallium during early development. They migrate all the way to the dorsal telencephalon where they make local synaptic connections with excitatory glutamatergic neurons and critically regulate local brain circuitry by releasing the inhibitory neurotransmitter GABA (Bandler et al., 2017; Wonders and Anderson, 2005). Compromised function of cINs is associated with various brain disorders such as schizophrenia, autism and epilepsy (Marin, 2012; Zhu et al., 2018). Further understanding of the cIN-associated disease pathogenesis mechanism critically depends on securing relevant tissue sources for analysis. Obtaining relevant brain tissues during the pathogenesis process was once extremely challenging except for the end disease stage postmortem tissues or rare resected patient brain tissues. However, now it is possible with the development of induced pluripotent stem cell (iPSC) technology that can generate disease relevant tissues in unlimited quantity with the exactly same genetic makeup as the patient (Takahashi and Yamanaka, 2006), as long as efficient ways to generate a homogeneous population of specific progenies can be identified.

Not only providing clues to normal and abnormal developmental process in health and disease, human PSC-derived neurons can provide cells for cell replacement therapy, allowing treatment of brain disorders for patients without other effective treatment options (Goldman, 2016; Kikuchi et al., 2017; Liu et al., 2013; Rhee et al., 2011; Southwell et al., 2014; Tabar and Studer, 2014; Zhao et al., 2017). In particular, iPSC-derived neuronal progenies could provide autologous cell sources for cell replacement without the need of immunosuppression or concern of immune rejection. Cell transplantation, especially transplantation of cINs, was shown to provide improvement of symptoms in diverse preclinical models of CNS disorders such as epilepsy, Parkinson's disease, neuropathic pain, schizophrenia and cognitive deficits (Braz et al., 2012; Cunningham et al., 2014; Donegan et al., 2017; Liu et al., 2013; Martinez-Cerdeno et al., 2010). Full realization of these promising preclinical studies into clinical reality critically depends on the ability to generate large quantities of homogeneous cell populations that are in the optimal stage of development for therapeutic use. Too immature progenitor cells will not migrate and integrate into the host circuitry well and may continue to proliferate, whereas too mature cells could be too fragile for passaging and transplantation and also will not migrate as well as embryonic cINs.

Medial ganglionic eminence (MGE) progenitors and cINs have been derived from hPSCs either by activating the developmentally relevant signaling pathways during differentiation (Kim et al., 2014; Liu et al., 2013; Maroof et al., 2013; Nicholas et al., 2013) or by direct induction using exogenous expression of fate-inducing transcription factors (Sun et al., 2016; Yang et al., 2017). However, large scale generation of cINs that can meet the demand of translational and clinical use for disease modeling/drug screening/cell therapy have not been demonstrated. Protracted maturation of human cINs require more time in culture to achieve proper maturation for disease modeling or cell therapy. Most of these methods involve coculture with other cell types such as astrocytes or glutamatergic neurons to support long-term maturation and maintenance in vitro (Colasante et al., 2015; Maroof et al., 2013; Nicholas et al., 2013; Sun et al., 2016), yielding mixtures of cell populations with variable functional properties. This is not optimal for transcriptome analysis or cell therapy use and awaits a more homogeneous culture system that supports long-term culture. In addition, hPSC-derived neuronal progenies are usually asynchronous, composed of proliferating progenitors and postmitotic neurons at the same time just as during normal development and such stochasticity and heterogeneity always raise the concern of unreliable assay for disease modeling (Hoffman et al., 2017) and graft safety for cell therapy (Amariglio et al., 2009; Berkowitz et al., 2016). Protracted maturation of the cIN itself is a hurdle in efficient use of cINs for various assays, raising the cost and time required before attaining reasonable maturation status for utilization and assay. This requires development of a method that can facilitate the maturation of cINs, preferably without genetic modifications that pose the risk of insertional mutagenesis that can compromise both disease study and cell therapy.

Accordingly, novel methods for generation and cryopreservation of synchronized cINs and are highly desirable.

### SUMMARY OF THE INVENTION

The present disclosure relates to optimized methods for generation of synchronized cINs population of cells derived from pluripotent stem cells. In one aspect, the disclosure relates to an optimized spinner culture method for generation of three-dimensional (3D) cIN spheres that supports industrial scale generation of homogeneous cIN populations. The disclosed feeder-free culture system sustained generated cINs in the long term without compromising their survival or phenotype and was optimized for passaging and cryopreservation with the incorporation of Trehalose. Moreover, their synchronized maturation was optimized into early postmitotic migratory cINs using a cocktail of chemicals, referred to herein as CDP. Their maturation was demonstrated in terms of metabolism, migration, arborization, and electrophysiology. As disclosed herein chemical treatment of hPSC-derived MGE cells was efficient in controlling proliferation in the grafts after transplantation into Nod Scid mice brains and promoting migration and integration of grafted cells in the host brains. The present disclosure provides transplantation-ready homogeneous populations of synchronized cINs of proper developmental stage for optimal grafting.

### BRIEF DESCRIPTION OF THE DRAWINGS

FIG. 1A-F. Optimization of large-scale generation of MGE cIN progenitors. FIG. 1A. Scheme of MGE progenitor phenotype induction from hPSCs. White scale bar=5 mm. Yellow scale bar=200 μm. FIG. 1B. Spinner culture efficiently generated MGE progenitor populations in large quantities. Spheres under different culture conditions were trypsinized at the end of week 3 for cell counting. For the comparison of relative fold changes in total cell numbers, one-way ANOVA (p<0.001) followed by Tukey post-hoc analysis was performed (Table S3). For the comparison of sphere sizes, one-way ANOVA (p<0.001) followed by Tukey post-hoc was performed (Table S3). Data are presented as mean±SEM (n=6−9 independent differentiation) FIG. 1C-D. Spinner culture maintained the MGE progenitor phenotype. Cells were plated onto coverslips after 3 weeks' differentiation and analyzed for the MGE progenitor phenotype (NKX2.1⁺). Scale bar=50 μm. Data are presented as mean±SEM (n=3 independent differentiation). For the comparison of NKX2.1⁺ cells, one-way ANOVA (p=0.171) was performed. FIG. 1E-F. Generated MGE progenitors under different conditions generated cINs 6 weeks after differentiation. Scale bar=50 μm. Data are presented as mean±SEM (n=3 independent differentiation). For the comparison of cINs markers, one-way ANOVA was performed for SOX6 (p=0.174), GAD (p=0.708), and β-TUBLIN (p=0.541).

FIG. 2A-E. Long-term culture and passaging of cIN organoids. FIG. 2A. Long-term culture scheme for the cIN organoids. FIG. 2B. Immunohistochemistry analysis of H9 cIN organoids after four weeks' differentiation for MGE progenitor phenotype (NKX2.1, NESTIN, and SOX2) and cIN phenotype (DLX). Scale bar=100 μm. FIG. 2C. Immunohistochemistry analysis of cIN organoids after twenty-four weeks' differentiation for the MGE progenitor marker (NKX2.1), cIN markers (GABA, β-TUBLIN, DLX) and early postmitotic neuronal marker (DCX). Scale bar=100 μm. FIG. 2D. Real time PCR analysis of 4 weeks old vs 40 weeks old cIN organoids. Data are presented as mean±SEM (n=3 independent differentiation). For the comparison of gene expression, one-way ANOVA was performed for KI67 (p=0.027), GAD (p=0.048), SOX6 (p=0.003), DLX (P<0.001), LHX6 (p=0.022), and SST (p=0.001). The Tukey post-hoc analysis was summarized in Table S4. FIG. 2E. Trehalose efficiently increased the viability of the cINs during organoid passaging. Data are presented as mean±SEM (n=4 independent differentiation) using a two-tailed unpaired t-test (p=0.006 for 6 w and p=0.037 for 24 w).

FIG. 3A-F. Combined chemical treatment significantly reduce proliferating progenitor cells in cINs culture FIG. 3A. Immunohistochemistry analysis of KI67⁺ proliferating progenitors in cIN organoids 12 weeks and 24 weeks after differentiation. White scale bar=100 μm, Yellow scale bar=50 μm. FIG. 3B. Chemical treatment scheme for MGE progenitors. FIG. 3C-D. Immunocytochemistry and cell counting analysis of KI67⁺ proliferating MGE progenitors after treatment with different chemicals for 1 week. Scale bar=50 μm. For comparison of the proportion of proliferating cells, one-way ANOVA (p<0.001), followed by Tukey post-hoc analysis was performed (Table S5). Data are presented as mean±SEM (n=4 independent differentiation). FIG. 3E. Immunocytochemistry analysis of 6 weeks old cINs treated with CDP for 0, 1, and 3 weeks. Scale bar=50 μm. FIG. 3F. Cell counting analysis. One-way ANOVA (p=0.001), followed by Tukey post-hoc analysis showed significant decrease of KI67⁺ cells after CDP treatment for one week (p<0.001) and three weeks (p<0.001) compared to the control group (Table S6). One-way ANOVA showed no significant difference among groups for SOX6⁺ cells (p=0.985), GAD⁺ cells (p=0.931), β-TUBLIN⁺ cells (p=0.896) and CASPASE-3⁺ cells (p=0.789). Data are presented as mean±SEM (n=5 independent differentiation).

FIG. 4A-C. CDP treatment facilitates metabolic maturation of cINs. FIG. 4A. DAVID analysis of DE genes between purified mouse cINs from E13.5 vs. cINs from adult brain, showing significant changes in Metabolism pathway. FIG. 4B. Analysis scheme for the metabolic maturation of cINs after CDP treatment. FIG. 4C. CDP treatment significantly enhanced the metabolic maturation of cINs. Data are presented as mean±SEM (n=10 wells) using paired one-way ANOVA. The Tukey post-hoc analysis was listed in Table S7.

FIG. 5A-D. CDP treatment enhances migratory, morphological, and electrophysical maturation. FIG. 5A. Analysis scheme for migration, arborization and electrophysiology of cINs. FIG. 5B. CDP treatment significantly increased migration of generated iPSC cINs. cIN organoids were embedded in a Geltrex matrix at 9 weeks of differentiation with or without CDP treatment, and analyzed for migration 7 days after embedding. White scale bar=200 μm and Yellow scale bar=100 μm. Data are presented as mean±SEM (n=3 independent spheres) using a two-tailed unpaired t-test (p=0.019 for cells in the spheres, p=0.008 for cells with migration distance of 0-400 μm, and p=0.001 for cells with migration distance >400 μm). FIG. 5C. CDP treatment significantly enhances arborization of H9 cINs. Neurites numbers from soma (p=0.180), Branch numbers (p=0.001) and Neurites lengths (p=0.005) were analyzed by two-tailed unpaired t-test (n=12 neurons). Data are presented as mean±SEM. FIG. 5D. CDP treatment significantly enhanced the electrophysiological maturation of cINs after nine weeks' CDP treatment. Data are presented as mean±SEM (n=24 control neurons and n=28 CDP-treated neurons) using a two-tailed unpaired t-test for resting membrane potential (RMP, p=0.041), membrane resistance (Rm, p=0.001) and membrane capacitance (Cm, p<0.001). CDP treatment generated a higher proportion of neurons with action potential firing (using Chi-square test, p=0.001) with significant increase of AP threshold (p=0.046).

FIG. 6A-G. Transplantation analysis of CDP-treated cINs after transplantation into Nod Scid mice cortex FIG. 6A. Scheme of Transplantation analysis. cINs with or without one week's CDP treatment were transplanted into the cortex of Nod Scid mice and grafts were analyzed one month after transplantation. FIG. 6B-C. Untreated or CDP-treated H9 cells generate grafts enriched with MGE-type cINs, as shown by immunohistochemistry analysis. White scale bar=50 μm and yellow scale bar=25 μm. FIG. 6D. CDP treatment cINs generated grafts with a lower proportion of proliferating cells, as analyzed by immunohistochemistry using antibodies against human NCAM and KI67. The white dotted lines mark the graft borders. Scale bar=100 μm. FIG. 6E. CDP treatment significantly decreased the proportion of proliferating cells in the graft one month after transplantation (p<0.001) and total graft cell numbers (p=0.049). Data are presented as mean±SEM (n=4) using a two-tailed unpaired t-test. FIG. 6F. Migration analysis of grafted cINs. Data are presented as mean±SEM (n=3) using a two-tailed unpaired t-test; (p=0.002 for cells in graft core, p=0.008 for cells with migration distance 0-100 μm and p=0.001 for migration distance >100 μm). FIG. 6G. Inhibitory GABAergic synapse analysis of grafted cINs one month after transplantation. Scale bar=5 μm. Data are presented as mean±SEM (n=5) using a two-tailed unpaired t-test (p<0.001).

FIG. 7A-F. Cryopreservation of MGE progenitors. FIG. 7A. Analysis scheme of MGE progenitor cryopreservation. FIG. 7B. Trehalose in freezing media increased the H9 cINs cell survival during cryopreservation. cINs progenitors were trypsinized after three weeks' differentiation and the same number of cells were frozen with or without Trehalose in freezing-media. Total cell numbers were counted after three weeks' recovery from cryopreservation as a sphere culture. Data are presented as mean±SEM (n=4 independent differentiation) using a two-tailed unpaired t-test (p<0.001). FIG. 7C-D. Trehalose treatment during the freeze-thaw cycle did not alter the cIN phenotype. The cINs were analyzed three weeks after thawing by immunocytochemistry for cIN marker expression (SOX6, GAD, and β-TUBLIN). Scale bar=50 μm. Data are presented as mean±SEM (n=4 independent differentiation) using a two-tailed unpaired t-test; (SOX6, p=0.797; GAD, p=0.128; β-TUBLIN, p=0.063). FIG. 7E-F. The optimized cryopreservation protocol maintained cIN phenotype well compared to the cells without cryopreservation. The cINs, three weeks after thawing, were analyzed for the cIN phenotypes (SOX6, GAD, and β-TUBLIN) by immunocytochemistry. Scale bar=50 μm. Data are presented as mean±SEM (n=4 independent differentiation) using two-tailed unpaired t-test; SOX6, p=0.743; GAD, p=0.765; β-TUBLIN, p=0.391.

FIG. 8A-D. Phenotype analysis of cINs generated in large scale using spinner culture. FIG. 8A. Overall scheme of cIN differentiation. FIG. 8B-D. The H9 cINs induced under three different culture conditions were plated onto the coverslips after three weeks' differentiation and phenotypes were analyzed at the end of six weeks by immunocytochemistry (SOX6⁺, GAD⁺, and GABA⁺). Scale bar=50 μm. Data are presented as mean±SEM (n=3 independent differentiation) using one-way ANOVA, (SOX6, p=0.443; GAD, p=0.116; β-TUBLIN, p=0.234.

FIG. 9A-B. Phenotype analysis of cINs generated by spinner culture. FIG. 9A. H9 cINs generated by spinner culture were analyzed after 8 weeks' differentiation by immunocytochemistry (5-HT, TH and glutamate). Yellow scale bar=100 μm, and white scale bar=50 μm. Cell counting data are presented as mean±SEM (n=3 independent differentiation). FIG. 9B. cINs generated by spinner culture were analyzed after 12 weeks' differentiation by immunocytochemistry (SST and MEF2C). Yellow scale bar=100 μm, and white scale bar=50 μm. Data are presented as mean±SEM (n=3 independent differentiation).

FIG. 10. Phenotypes of long-term cIN organoid culture. H9 cINs organoids generated by spinner culture were analyzed after 24 weeks' culture by immunocytochemistry (NESTIN and active CASPASE3). White scale bar=100 μm.

FIG. 11A-B. Decreased total cell numbers in CDP-treated cINs. FIG. 11A. Phase Contrast microscopy analysis showed lower cell density after one week's CDP treatment. Same number of H9 cIN progenitors were plated onto the coverslips after 3 weeks' differentiation and analyzed 1 week after treatment. FIG. 11B. Cell counting analysis at 1 week after CDP treatment. A two-tailed unpaired t-test was used with p=0.001 (n=4). Data are presented as mean±SEM (n=4).

FIG. 12A-B. Metabolic maturation of cINs. FIG. 12A. Heat map depicting massive upregulation of metabolic genes in adult mice cINs compared with E13.5 cINs. Each number indicates log-fold changes of gene expression. Red color depicts increase in expression in adult cINs. FIG. 12B. Analysis scheme for oxidative phosphorylation using Seahorse analyzer. Oxygen consumption rate (OCR) were monitored through sequential injections of oligomycin, FCCP and rotenone/antimycin. Basal Respiration=baseline OCR-Rotenone/antimycin A OCR, ATP production=Baseline OCR-Oligomycin OCR, Maximum Respiration=FCCP OCR-Rotenone/antimycin A OCR, Space capacity=Maximum Respiration-Basal Respiration. The result was normalized to total protein levels quantified using a BCA protein assay (Thermo Fisher, Cambridge, Mass., US).

FIG. 13A-C. CDP treatment significantly enhanced morphological and electrophysiological maturation of generated cINs. FIG. 13A. Tracing of untreated or CDP-treated H9 cINs. cINs, infected with limiting titer of GFP-expressing lentivirus, were plated as an adherent culture after 3 weeks' differentiation and treated with or without CDP for 3 weeks, and traced for arborization analysis. Scale bar=50 μm. FIG. 13B. CDP treatment did not alter half-width of action potential (half-width of AP, p=0.353) nor afterhyperpolarization (AHP, p=0.978) of 12 weeks old cINs (n=24 control neurons and n=28 CDP-treated neurons). A two-tailed unpaired t-test was used and data are presented as mean±SEM. FIG. 13C. CDP treatment facilitated electrophysiological maturation of cINs analyzed after 6 weeks' differentiation. Spheres were trypsinized at the end of week 3 and treated with or without CDP for 3 weeks, followed by electrophysiology analysis at the end of 6 weeks. Data are presented as mean±SEM (n=18 control neurons and n=15 CDP-treated neurons) using a two-tailed unpaired t-test; Resting membrane potential (RMP, p=0.002; Rm, p=0.025; Cm, p=0.049).

FIG. 14. Inhibitory GABAergic synapse analysis of grafted cINs. Representative inhibitory synapse analysis of grafted H9 cINs one month after transplantation using IMARIS software. Scale bar=5μ

### DETAILED DESCRIPTION OF THE INVENTION

The present disclosure provides cell culturing methods for generating a population of synchronized cortical interneurons derived from pluripotent stem cells.

The types of pluripotent stem cells that may be used include established lines of pluripotent cells. Non-limiting examples are established lines of human embryonic stem cells or human embryonic germ cells, such as, for example the human embryonic stem cell lines H1, H7, and H9 (WiCell). Also contemplated is use of pluripotent stem cells derived from the host to be treated, e.g., autologous stem cells. Alternatively, the pluripotent stem cells may be allogenic stem cells derived from a donor that is determined to be an acceptable match to the patient to be treated. Also suitable are pluripotent stem cells derived from non-pluripotent cells, such as, for example, an adult somatic cells.

In one aspect of the invention, said method of generating a population of cortical interneurons comprises the initial method of generating MGE progenitors from pluripotent stem cells said method comprising the step of establishing sphere cultures from pluripotent stem cells under spinner culture conditions during MGE phenotype induction. For long term culture (more than about 6 weeks), a switch is made to static culture, since older and bigger spheres tends to break down under spinner culture condition. The subsequent generation of cINs from the established MGE progenitors comprises the step of culturing the MGE progenitor cells in the presence of one or more of the following components: CultureOne, gamma secretase inhibitor DAPT and/or CDK4/6 inhibitor PD0332991. In a specific embodiment, the culturing is done in the presence of all three components (referred to herein as CDP). In one aspect, components of CDP include, for example, 1% CultureOne (“C”, Thermo Fisher, Waltham, Mass., USA), 10 μM DAPT (“D”, Sigma-Aldrich, Natick, Mass., USA), and 2 μM PD0332991 (“P”, Sigma-Aldrich, Natick, Mass., US). In non-limiting aspects, for culturing of cINs, the concentrations of the DAPT component may be used in a range of about 3-10 μM; concentrations of the PD0332991 component may be used in a range of about 1-8 μM and concentrations of the CultureOne component may be used in the range of 1% -10%. The present disclosure is based, at least in part, on the observation that the addition of one or more components of CDP can increase the efficiency of cIN maturation.

In yet another aspect, the pluripotent stem cell derived cINs are grown in the presence of Trehalose during passaging. In another aspect, the cells are cryopreserved in the presence of Trehalose. Concentrations of Trehalose to be used may be between about 0.1-0.4M. In a specific embodiment, the concentration of Trehalose is about 0.1M. Said cINs, derived from pluripotent stem cells, may be characterized by the expression of markers, including one or more of the following markers, SOX6+, GAD+, DLX2+, B-tubulin+, and/or NKX2.1 depending on developmental age of the cINs. Additionally, one or more of the following markers, Parvalbumin, SST, LHX6 and/or MEF2C may be expressed on the derived cortical interneurons.

The present disclosure provides synchronized populations of cINs, derived from pluripotent stem cells using the culturing methods disclosed herein. In one aspect, the disclosure provides pharmaceutical compositions comprising the cINs, derived using the methods disclosed herein, and one or more pharmaceutical acceptable carriers and or excipients.

The present invention further provides, the use of the cortical interneurons described herein for transplantation into hosts in need of treatment. In one embodiment, the patient is in need of treatment for a brain disorder. In a specific, non-limiting embodiment, the brain disorder may be schizophrenia, autism and epilepsy.

Other embodiments of the present invention will be apparent to those skilled in the art from consideration of the present specification and practice of the present invention disclosed herein. Publications cited throughout this document are hereby incorporated by reference in their entirety. It is intended that the present specification and examples be considered as exemplary only with a true scope and spirit of the invention being indicated by the following claims and equivalents thereof.

The following examples are presented to further illustrate selected embodiments of the present invention.

### EXAMPLE

### Materials and Methods

Culture of hESCs and Differentiation into cINs Human embryonic stem cell (hESC) line H9 (WiCell, Madison, Wis., US, passage 30-50) was maintained on Geltrex (Thermo Fisher, Waltham, Mass., USA)—coated plates in Essential 8 Medium (Thermo Fisher, Waltham, Mass,, USA). ROCK inhibitor (Y27632, 10 μM, ApexBio, Boston, Mass,, USA) was added to the culture for 24 hours after passaging to prevent single cell-induced cell death of hESCs. All the chemicals used in this study were listed in Table S1.

MGE differentiation was initiated by passaging H9 cells as spheres in low adherent flasks with SRM media (DMEM with 15% knockout serum replacement (KSR), 2 mM L-glutamine and 10 μM β-mercaptoethanol (all from Thermo Fisher, Waltham, Mass., USA)). ROCK inhibitor was also included on the day of differentiation. For the first week, cells were differentiated in SRM LSsgW media (the SRM media supplemented with 0.1 μM LDN193189 (Selleck Chem, Houston, Mass., USA), 10 μM SB431452 (Tocris Bioscience, Minneapolis, Minn., US), 0.1 μM SAG (Selleck Chem, Houston, Mass., USA) and 5 μM IWP2 (Selleck Chem, Houston, Mass., USA)). For the second week, SRM Lsg media (the SRM media was supplemented with 0.1 μM LDN193189 and 0.1 μM SAG) was used. In the third and fourth weeks, the media was changed to N2AA media (DMEM/F12 media with 1% N2 supplement (Life Technologies, Woburn, Mass., USA) and 200 μM AA-Ascorbic acid (Sigma-Aldrich, Natick, Mass., USA)). For the third week, the N2AA media was supplemented with 1 μM SAG and 50 ng/ml FGF8 (ProSpect, Rocky Hill, Conn., USA). At the beginning of the fourth week, the N2AA media was supplemented with 5 ng/ml GDNF (ProSpect, Rocky Hill, Conn., USA) and 5 ng/ml BDNF (ProSpect, Rocky Hill, Conn., USA) (N2AAGB media). From the fifth week, the cells were maintained in the B27GB media (DMEM/F12 media with 1% B27 supplement (Thermo Fisher, Waltham, Mass., USA), 5 ng/ml GDNF and 5 ng/ml BDNF). For comparison of different culture conditions, cells were cultured in the flask as a static culture or shaking on orbital shaker at 80 rpm (SK-O180-E Analog Orbital Shaker, Scilogex, Rocky Hill, Conn., USA) or using stirrer culture system at 80 rpm (Celstir spinner flask (Wheaton, Millville, N.J., USA) and Multistirrer Digital Series Magnetic Stirrers (VELP Scientifica Srl, MB Italy)). All cell lines were routinely tested for mycoplasma once a week using a Mycoplasma Detection Kit (InvivoGen, San Diego, Calif., USA).

For passaging cINs, cells were trypsinized by 0.05% trypsin (Thermo Fisher, Waltham, Mass., USA) supplemented with or without 10 mM Trehalose (Sigma-Aldrich, Natick, Mass., USA). After 5 minutes' incubation at 37° C., the spheres were triturated to dissociate the spheres. An equal volume of DMEM media with 10% FBS (Hyclone, Marlborough, Mass., USA) was added to neutralize Trypsin. In addition, Turbo DNase (2 U/ml, Thermo Fisher, Waltham, Mass., USA) was added to help clear released DNAs from culture. The cells were then kept in the incubator for additional 15 min to remove any sticky DNA mass released during trituration. After incubation, the cells were centrifuged and resuspended by B27GB media with ROCK inhibitor. To exclude the dead cell cluster, the resuspended cells were filtered through a cell strainer cap (35 μm nylon mesh, Corning, N.Y., USA), and plated onto a PLO/FN-coated surface (Poly-L-Ornithine, 15 ug/ml, Sigma-Aldrich, Natick; Fibronectin, 10 ug/ml, Thermo Fisher, Waltham, Mass., USA) for the subsequent experiments.

For chemical treatment of cINs, the differentiated progenitors were seeded on PLO/FN-coated surface at 3 weeks after differentiation, and cultured in B27GB media without or with each chemical, 20 μM FdU (Sigma-Aldrich, Natick, Mass., USA), 1% CultureOne (C, Thermo Fisher, Waltham, Mass., USA), 10 mM DAPT (D, Sigma-Aldrich, Natick, Mass., USA), and 2 μM PD0332991 (P, Sigma-Aldrich, Natick, Mass., US), or with combination of CultureOne, DAPT and PD0332991 (CDP) for the time period designated in each Figure.

For cryopreservation, the trypsinized cells were resuspended in freezing media (FBS with 10% DMSO) with or without 100 mM Trehalose and were frozen slowly overnight in an insulated container at −80° C. deep freezer. After 24 hours, the stocks were moved to liquid nitrogen storage until further experiments. Frozen stocks were thawed quickly in a 37° C. water bath, transferred to 15 ml conical tubes with 5 ml media, centrifuged and resuspended in B27GB media with ROCK inhibitor for plating and further experiments.

Immunocytochemistry and cell counting. cINs on coverslips were fixed using 4% paraformaldehyde (PFA, Electron Microscopy Sciences, Hatfield, Pa.) for 15 min, washed with PBS and used for staining. The spheres at different differentiation stages were fixed by 4% PFA for 15 min, rinsed with PBS, cryo-protected in the 30% sucrose (Thermo Fisher, Waltham, Mass., USA) overnight at 4° C., mounted on the chuck using the O.C.T. Compound (Thermo Fisher, Waltham, Mass., USA) and cryosectioned at 40 μm using a Leica CM1850 cryostat (Leica Biosystem, Buffalo Grove, Ill., USA). Fixed cells or sphere sections were incubated with blocking/permeablization buffer (PBS with 10% normal serum and 0.1% Triton X-100) for 10 min. The samples were then incubated in primary antibodies in antibody dilution buffer (PBS containing 2% normal serum) overnight at 4° C. The detailed information of the antibodies used were listed in Table S2. After washing with PBS, cells were incubated with fluorescently labeled secondary antibodies and DAPI (Invitrogen, Waltham, Mass., USA)) in antibody dilution buffer for 1 h at room temperature. Following the PBS wash, the samples were mounted with Fluoromount-G (SouthernBiotech, Birmingham, Ala., USA).

Fluorescent images were taken by the EVOS FL Auto microscope (Life Technologies, Carlsbad, Calif.), Olympus DSU Spinning Disc Confocal on an IX81 inverted microscope (Olympus, Center Valley, Pa., USA) and Zeiss LSM710 Confocal Laser Scanning Microscopes (Zeiss, Oberkochen, Germany).

For cell counting, multi point function in Image J software (Version 1.51 p, NIH, Bethesda, Md., USA) was used. Percentage of positive cells for each marker was calculated by dividing by DAPI-stained total nuclei number from at least 3 separate biological replicates. For each staining, a total of at least 500 cells were counted for each group.

RNA preparation, Reverse transcription and real time PCR analysis. Cells were harvested using TRIzol (Thermo Fisher, Waltham, Mass., USA), and total RNAs were prepared according to the manufacturer's protocol. For the reverse transcription, 500 ng total RNA was first reversely transcribed to cDNA using RevertAid H Minus Reverse Transcriptase (Thermo Fisher, Waltham, Mass., USA). The real-time PCR reaction was carried out in a 96-well format with SsoAdvanced™ Universal SYBR® Green Supermix (Bio-Rad, Hercules, Calif., USA). The primer information is as follows: KI67 (F 5′-TCCTTTGGTGGGCACCTAAGACCTG, R 5′-TGATGGTTGAGGTCGTTCCTTGATG), GAD (F 5′-CTGCTCTTCTCTTACGCTCTCTGTC, R 5′-TCTTCGGAAATGTTGCCTTAGG), SOX6 (F 5′-ATCTCTCATCCCGACCCAAGAC, R 5′-TTCCCAGGCTTCCTCCAATG), DLX2 (F 5′-GCCTCAACAACGTCCCTTACT, R 5′-GGGAGCGTAGGAGGTGTAGG), LHX6 (F 5′-ATTCCTTGCGTGGATTATGTGG, R 5′-TCCGTGTGTGTGTTTTCCCC), SST (F 5′-CAGGATGAAATGAGGCTTGAGC, R 5′-TTAGGGAAGAGAGATGGGGTGTGG). All reactions were carried out and analyzed using the CFX96 Real-Time PCR System (Bio-Rad, Hercules, Calif., USA). The expression of GAPDH mRNA in each sample was used to normalize the data. Relative gene expression was analyzed by 2^{-ΔΔCt} method.

Identification of cIN maturation-specific differentially expressed (DE) genes. To identify transcriptome changes during cIN maturation, we compared the transcriptome of purified cINs in the E13 mice cortex (Faux et al., 2010) vs. purified cINs in the P40 mice cortex (Okaty et al., 2009). All MoGene 1.0 ST arrays (Faux et al., 2010) were processed using the ‘oligo’ BioConductor package (Carvalho and Irizarry, 2010) and all Mouse 430A2 arrays (Okaty et al., 2009) with the ‘affy’ Bioconductor package (Gautier et al., 2004). Both the E13 cortex and P40 interneurons' array data were quality-controlled with array Quality Metrics (Kauffmann et al., 2009), normalized with RMA (Irizarry et al., 2003), filtered, subset to one probe per gene and further subset to common genes between the two datasets. DE genes were identified using adjusted p value less than 10⁻⁸. Pathways enriched after cIN maturation were identified using DAVID (https://david.ncifcrf.gov/).

Oxydative phosphorylation analysis using seahorse analyzer. Mitochondrial activity of cINs was measured using the Seahorse XFp8 analyzer (Agilent Technologies, Santa Clara, Calif., USA) according to the manufacturer's instructions. Briefly, cells were plated in the XF cell culture miniplate and incubated at 37° C. with 5% CO2. One day before the test, the cartridge with XF calibrant was incubated in a non-CO2 incubator overnight to equilibrate. Before assay, the media was changed to XF assay medium supplemented with 5 mM sodium pyruvate (Thermo Fisher, Waltham, Mass., USA), 10 mM glucose (Thermo Fisher, Waltham, Mass., USA), and 2 mM glutamine, and equilibrated in a non-CO₂ incubator for 1 hour. Oxygen consumption rate (OCR) were monitored through sequential injections of 1 μM oligomycin, 0.3 μM FCCP and 1 μM rotenone/antimycin A (Seahorse XF Cell Mito Stress Test Kit, Agilent, Santa Clara, Calif., USA). Raw data were used to calculate the various parameters of the mitochondrial activity: Basal Respiration=baseline OCR-Rotenone/antimycin A OCR, ATP production=Baseline OCR-Oligomycin OCR, Maximum Respiration=FCCP OCR-Rotenone/antimycin A OCR, Oxidative Reserve=Maximum Respiration-Basal Respiration, H+ (Proton) Leak=Oligomycin OCR-Rotenone/antimycin A OCR and Space Capacity=Maximum Respiration-Basal Respiration. The result was normalized to total protein levels quantified using a BCA protein assay (Thermo Fisher, Cambridge, Mass., US).

cINs migration and arborization analysis in vitro. For in vitro migration analysis, 9 weeks old cIN spheres were embedded in Geltrex matrix and treated with or without CDP for 7 days. Phase pictures were taken on day 0 and day 7 after embedding to follow migration of cINs. For quantification of migrated cells, the embedded spheres were incubated with Hoechst (Sigma-Aldrich, Natick, Mass., USA) overnight on day 7, and the entire embedding was imaged and tiled for Hoechst signal, and used for cell counting using the multipoint function in Image J software. Cell numbers within sphere core, in migration distance 0-400 μm, and in distance longer than 400 μm were counted separately for each sphere.

For arborization analysis, the MGE progenitors were plated onto the PLO/FN-coated coverslips after three weeks' differentiation with or without CDP. Two weeks later, the MGE cells were infected with limiting titer (MOI=0.001) of LV-UbiC-GFP virus (Hong et al., 2007) to label cells only scarcely. One week after infection, the images of GFP⁺ cells were collected by the EVOS microscope. The arborization of each GFP⁺ cell was analyzed using Image J software with the Neuron J plugin to get parameters of total neurite length, total branch number, and neurite number from soma.

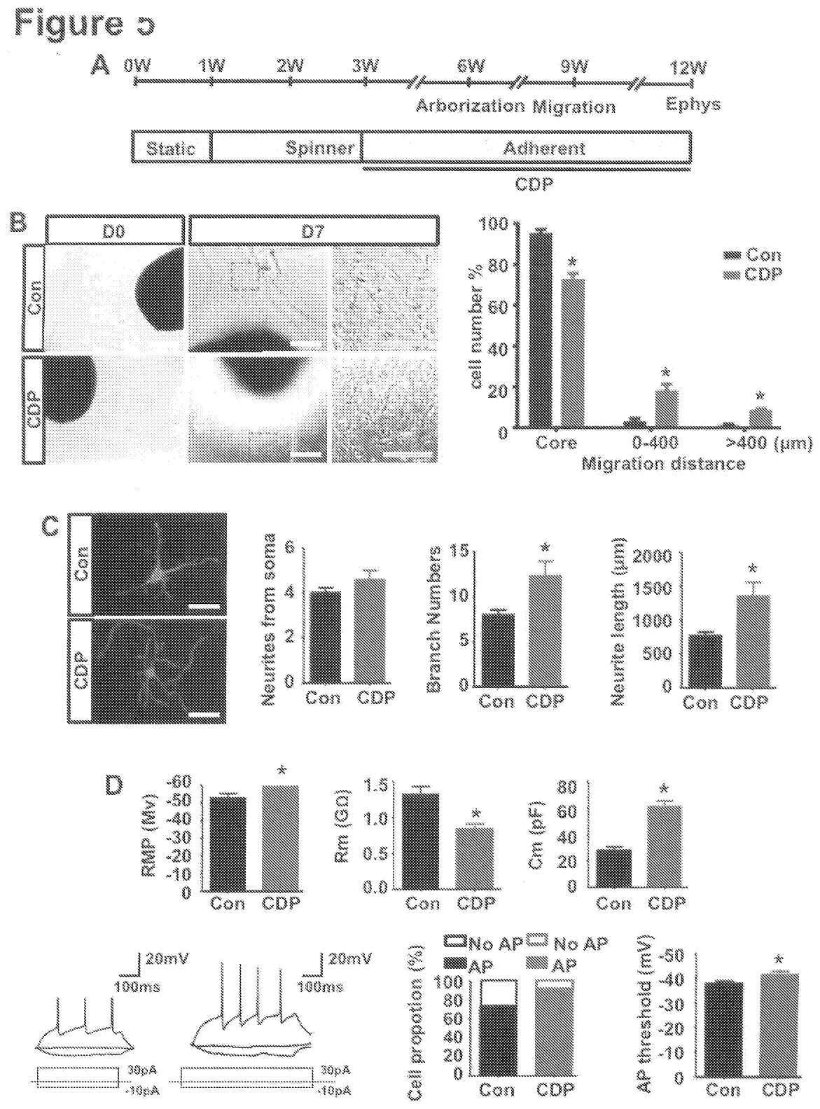

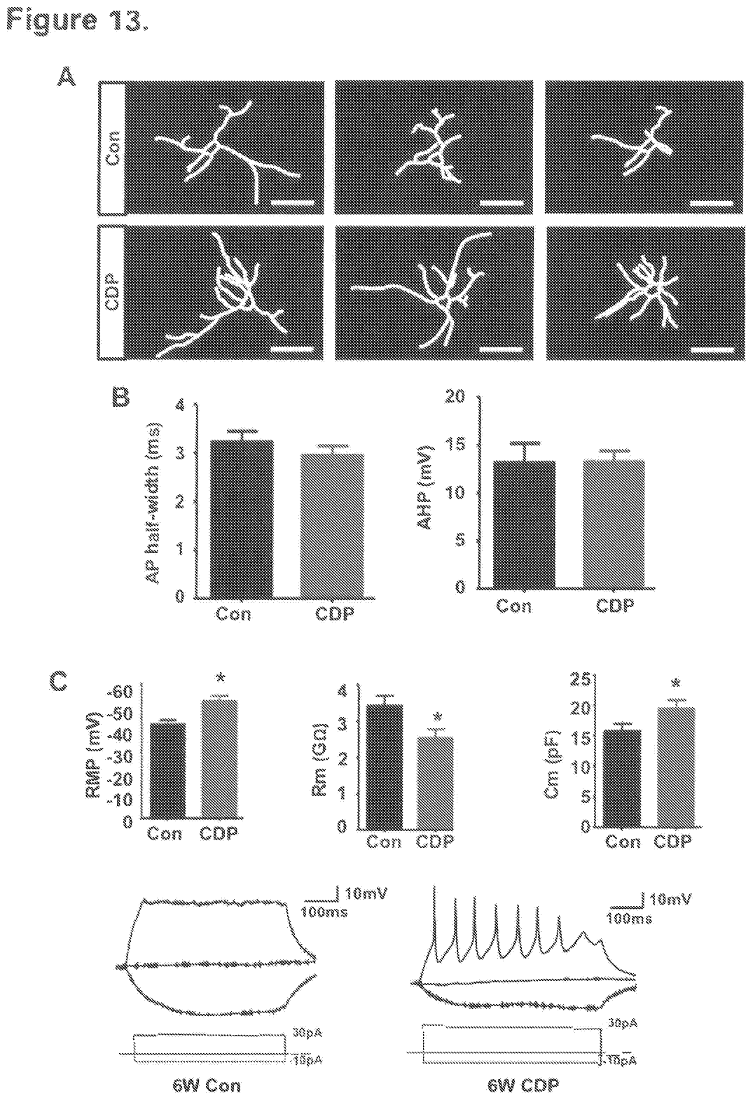

Electrophysiological analysis in vitro. Three weeks old MGE progenitor cells were cocultured on PF-coated coverslips with rat Cortical Astroglial Cells (PC36108, Neuromics, Edina, Minn., USA) at a ratio of 5:1 and in B27GB media supplemented with 1.8 mM CaCl₂. cINs were then infected with a LV-Syn-Chr2-YFP virus (Plasmid #20945, Addgene, Cambridge, Mass., USA), and maintained with or without CDP treatment for 9 weeks. At 12 weeks, cINs were transferred into a recording submersion chamber which was continuously perfused at room temperature (21-23° C.) at a flow rate of 1.5-2 ml/min with artificial cerebrospinal fluid (130 mM NaCl, 2.5 mM KCl, 2.5 mM CaCl₂, 1 mM MgSO₄, 1.25 mM NaH₂PO₄, 26 mM NaHCO₃, and 10 mM glucose) with an osmolarity of 295-305 mM, gassed continuously with 5% CO₂/95% O₂. Whole-cell patch-clamp recordings were acquired with a Multiclamp 700B, Digidata 1550, and Clampex 10 software (Molecular Devices). Whole-cell patch-clamp electrodes (3-5 MΩ resistance) were filled with an intracellular solution containing in mM:120 K-gluconate, 9 KCl, 10 KOH, 8 NaCl, 10 HEPES, 3.48 MgATP, 0.4 Na₃GTP, 17.5 sucrose, 0.5 EGTA, with an osmolarity of ˜290 and pH 7.3. Sampling was done at 5 kHz unless specified otherwise. Liquid junction potential of 9.7 mV was corrected for the internal pipette solution used. Resting membrane potential was recorded at I=0. To compare membrane excitability, membrane potential was held at −70 mV and square pulses of depolarizing current steps were applied in current clamp mode (−10 −80 pA in increments of 10 pA, 0.8 s duration). The first action potential (AP) induced by depolarizing current was used to analyze AP threshold, afterhyperpolarization amplitude (AHP), and AP half-width, using Clampfit software (Molecular Devices). Membrane capacitance was calculated with the membrane test function on Clampex 10, using a 5 mV voltage pulse from the holding voltage of −70 mV and recorded currents in voltage-clamp mode with a sampling rate of 100 kHz. Membrane resistance (Rm) was calculated in current clamp mode by applying a −10 pA current step and using the steady-state voltage deflection to calculate Rm as (steady-state voltage deflection/−10 pA). Averages±SEM are represented in FIG. 5 and FIG. 13B.

cINs were also splitted after 3weeks′ differentiation without feeder cells. After treated with or without CDP for 3 weeks, the electrophysiology analysis was performed as described above. Averages±SEM of these cells are represented in FIG. 13C.

Transplantation and immunohistochemistry analysis. All animal procedures were carried out in accordance with the approved guidelines and all animal protocols were approved by the Institutional Animal Care and Use Committee at New York Medical College. The cINs were trypsinized and plated on PLO/FN plate at the density of 10⁵/cm² at the end of three weeks' differentiation. After one week's CDP treatment, the cINs were trypsinized and resuspended in transplantation media (HBSS with 4.5 mg/ml sucrose, 10 ng/μl GDNF, 10 ng/μl BDNF, 20 nM Boc-Asp(OMe) fluoromethyl ketone (BAF, Sigma-Aldrich, Natick, Mass., US) and 10 μM Rock Inhibitor at a density of 1×10⁵/μl. A 1 μl volume of cINs were injected into the cortices of Nod Scid mice (Charles River Laboratory, Kingston, N.Y.) using a Kopf stereotaxic instrument (Kopf, Tujunga, Calif.) with a mouse adapter (Stoelting, Wood Dale, Ill.) under isoflurane anesthesia (3% induction, followed by 1% maintenance). The cells were injected at each of the following coordinates: AP 0.00 mm, L ±3.28 mm, V −1.80 mm; AP −2.12 mm, L ±4.20 mm, V −2.00 mm. One month after transplantation, the mice were perfused with 0.1 M PBS followed by 4% paraformaldehyde (PFA). The brains were removed, and post-fixed in 4% PFA solutions overnight and then placed in 30% sucrose solutions for one day. Forty-micrometer-thick coronal sections were cut on a Leica CM1850 cryostat (Leica Biosystem, Buffalo Grove, Ill., USA). The immunohistochemistry process followed the same procedure as immunocytochemistry as described above. The antibodies used are summarized in Table S2. Images were captured and analyzed by EVOS FL Auto microscope (Life Technologies, Carlsbad, Calif.) and Zeiss LSM710 Confocal Laser Scanning Microscopes (Zeiss, Oberkochen, Germany).

For in vivo migration analysis, hNCAM/DAPI images of areas including more than 500 μm around the graft were captured and tiled using Zeiss LSM710 Confocal Laser Scanning Microscopes with 10× objective, and grafted cell numbers were counted using the multi point function in Image J software. Grafted cells in the graft cores, in migration distance of 0-100 μm and in migration distance longer than 100 μm were counted separately for each graft.

For synapse analysis, images were captured using Zeiss LSM710 Confocal Laser Scanning Microscopes (Zeiss, Oberkochen, Germany) with 100× objective and processed using Imaris software (Bitplane, Switzerland), which allows objective counting of synaptic puncta based upon absolute fluorescent intensity. Synaptic puncta positive for VGAT (inhibitory presynaptic) and Gephyrin (inhibitory postsynaptic) were identified with spot diameters of 0.6 μm. For synapses, a juxtaposition of hNCAM⁺VGAT⁺ puncta and Vglut1⁺ puncta was determined as inhibitory synapses. The result was expressed as synapse numbers per human NCAM⁺ neuronal surface area (μm²). We analyzed a total of 795 neurite segments with a total area of 7442 μm² for control cINs and a total of 977 neurite segments with a total area of 7442 μm² for CDP-treated cINs. Total counted colocalized puncta numbers are 37 for control cINs and 161 for CDP-treated cINs.

Statistical analysis. The statistical analysis was performed using GraphPad Prism7 (GraphPad Software, La Jolla, Calif.). A two tailed, unpaired t-test was used to compare the difference between two groups. One-way ANOVA was used to compare the difference among multiple groups, and once a significant difference was observed, Tukey Post Hoc Test was performed to compare the difference between any two groups. When p<0.05, the difference was considered as statistically significant.

### RESULTS

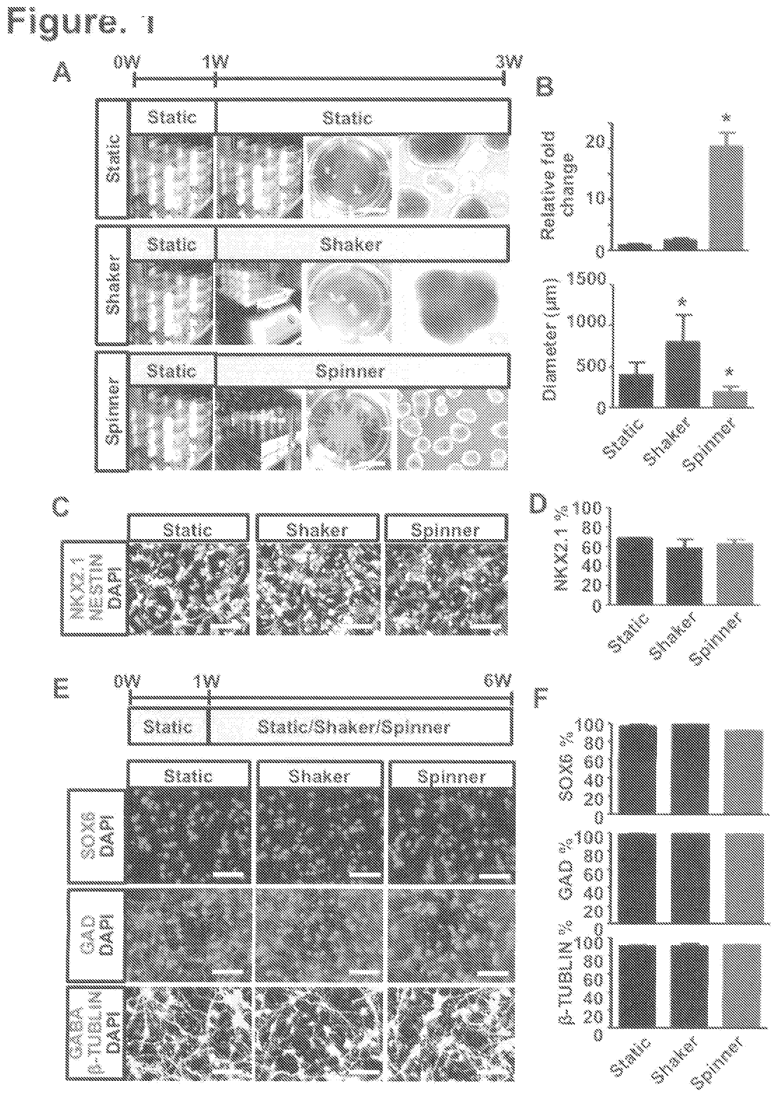

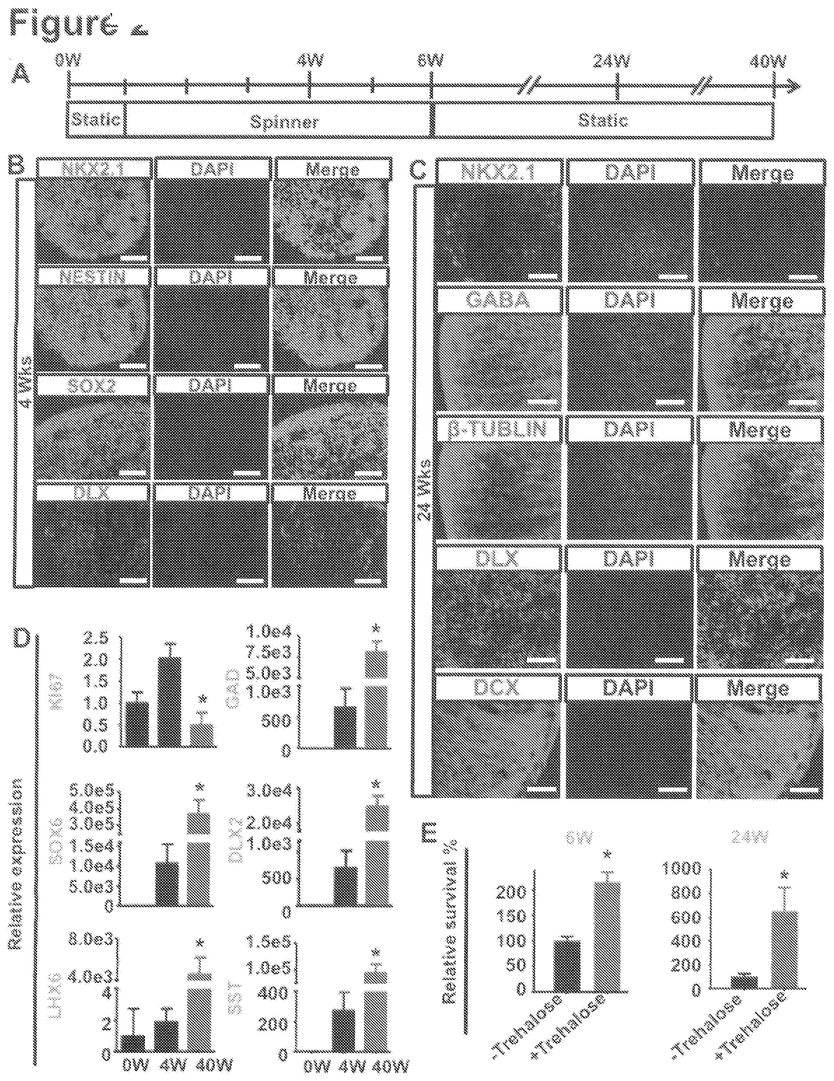

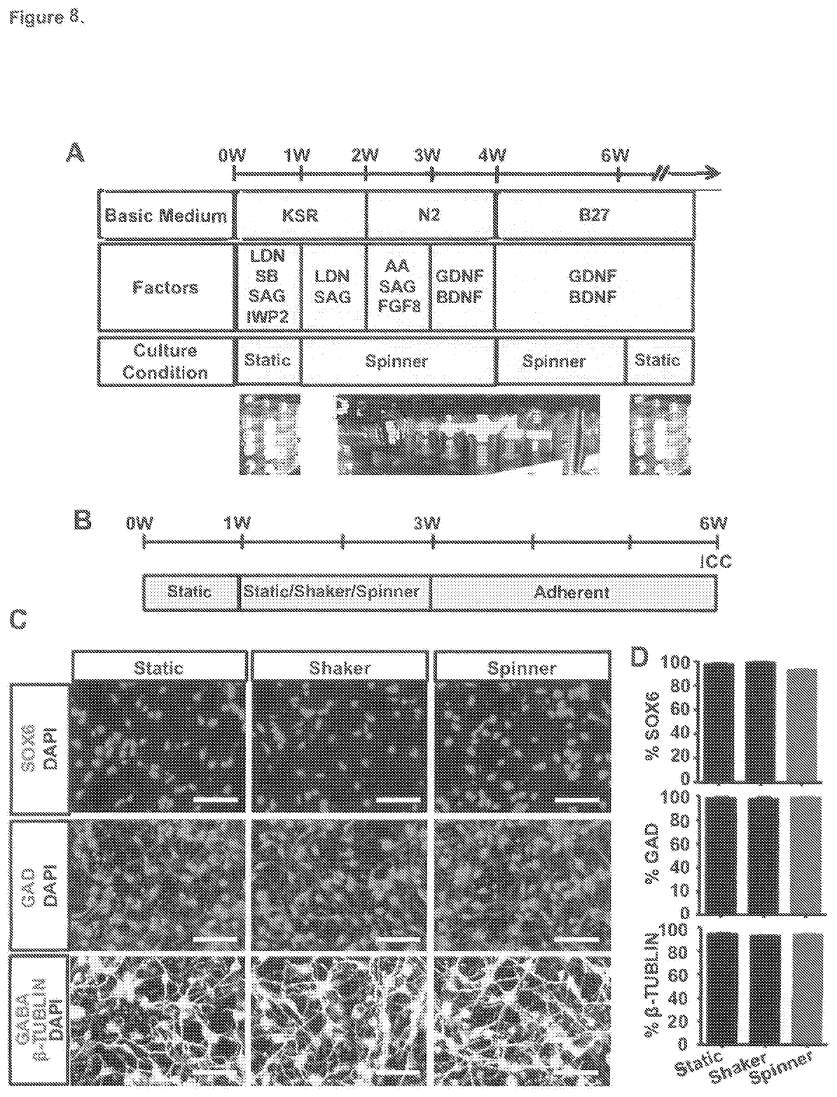

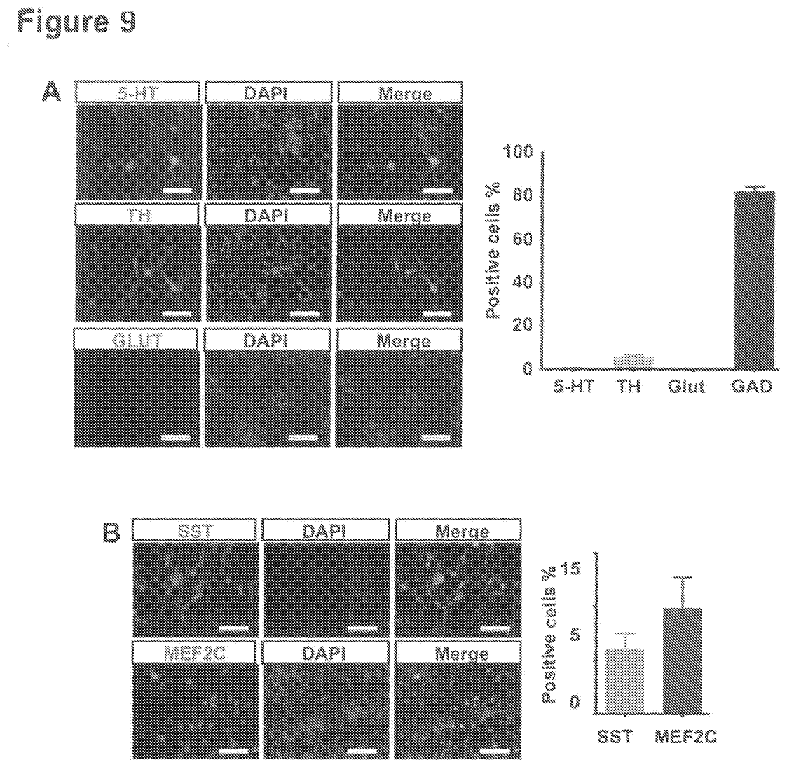

Optimization of large-scale generation of MGE cortical interneuron progenitors. A protocol was developed to generate homogeneous populations of cortical interneurons (cINs) from human pluripotent stem cells (hPSCs) based on ventral specification of sphere/organoid culture (Ahn et al., 2016; Kim et al., 2014). For efficient use of hPSC-derived cortical interneurons, it is imperative to derive them in a scale large enough for industrial use in drug screening or cell therapy. Thus, the effect of different methods of culture on expansion and generation of MGE progenitors was tested. The MGE progenitor cells were induced for three weeks according to the previous report (Kim et al., Ahn et al.) with slight modification (FIG. 8). After establishment of sphere cultures under static condition for a week, three different culture conditions (static, shaker and spinner) were tested for two weeks. Spinner culture generated significantly higher increases in total cell numbers compared to static and shaker cultures (FIG. 1A-B and Table S3). This is possibly due to more efficient air/nutrient/waste exchange in the spinner system. Spinner culture also presented more homogeneous spheres size, without random adhering of spheres that can cause spheres to become too big for efficient air/nutrient/waste exchange (FIG. 1A-B and Table S3). Next, the phenotype of generated cells in each culture condition was examined to rule out the possibility that spinner culture favors the induction/expansion of alternate phenotype cells with faster growth rate than MGE progenitors. Despite massive expansion, cells grown in spinner culture also showed a homogeneous MGE progenitor phenotype shown by uniform NKX2.1 expression (FIG. 1C-D). When further differentiated, cells from spinner culture generated homogeneous populations of cINs shown by uniform expression of SOX6, GAD and β-TUBLIN either as sphere cultures for six weeks (FIG. 1E-F) or after transferring to adherent cultures from three weeks till six weeks (FIG. 8B-D). There were very few other neuronal types generated such as serotonergic (5HT⁺), dopaminergic (TH⁺) or Glutamatergic (Glutamate⁺) (FIG. 9A). Some of the generated cINs started to express either SST or MEF2C, a recently identified prospective PV lineage cIN marker (FIG. 9B).

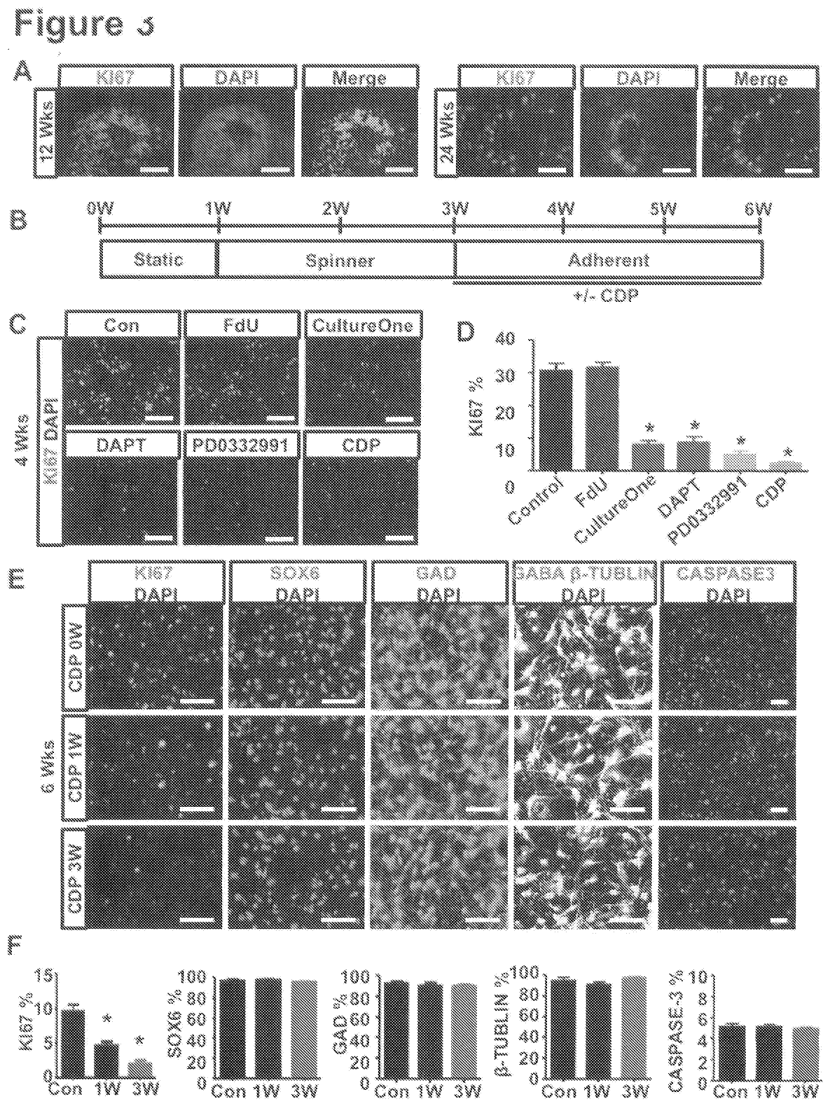

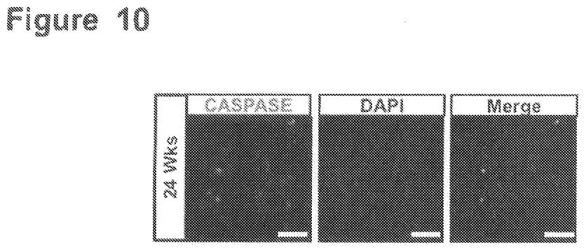

Long-term organoid culture of cINs. Long-term culture of cINs was previously achieved by coculturing with the feeder cells such as astrocytes and glutamatergic neurons (Colasante et al., 2015; Maroof et al., 2013; Nicholas et al., 2013; Sun et al., 2016). While feeder cells enable the long-term culture of generated human cINs, they may not be optimal for disease modeling or cell therapy. Thus, it was tested whether a homogeneous population of MGE cells could be maintained as cIN organoids on a long-term basis without compromising the viability of the cINs. MGE spheres were maintained as spinner cultures for 6 weeks and then transferred to static culture to avoid shearing of organoids (FIG. 2A). After four weeks of organoid culture, MGE progenitor markers such as NKX2.1, NESTIN and SOX2 (FIG. 2B) were highly expressed, whereas post-mitotic cIN marker DLX was relatively low. MGE organoids after 24 weeks of culture expressed less MGE progenitor marker NKX2.1 but more post-mitotic cINs markers such as GABA, β-TUBLIN, DCX and DLX and were still healthy without much sign of apoptosis (FIG. 2C and FIG. 10). The changes in gene expression level as the organoid matured was quantitatively analyzed by real time PCR. Significant decrease in the expression of proliferating cell marker KI67 was observed as cIN organoids matured, whereas the expression of post-mitotic cIN markers GAD, SOX6, DLX, LHX6 and SST were significantly higher in 40 week-old cIN organoids compared to 4 week-old organoids (FIG. 2D and Table S4).

One of the barriers of utilizing organoid culture for long-term maintenance of cINs is the difficulty of passaging older organoids, as they develop thick networks of neurites just as during normal brain development. Since it was reported that Trehalose could significantly improve the cell viability after dissociation of neurons in mature mouse brains (Saxena et al., 2012), the effect of Trehalose on the dissociation of tightly-knit older organoids was tested. Trehalose treatment during passaging significantly increased the survival rate 6 weeks after differentiation. The increase in survival rate was much more pronounced in older organoids, after six months' differentiation (FIG. 2E). Optimized passaging of older organoids will enable efficient utilization of cINs that are maintained long-term and feeder-free.

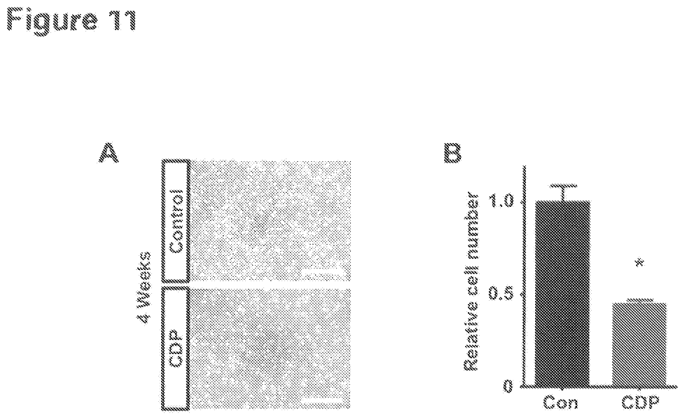

Combined chemical treatment significantly reduces proliferating progenitor cells in cINs culture. One thing noticed in long-term organoid culture was the presence of proliferating cells (KI67⁺) even 24 weeks after differentiation (FIG. 3A). This population of lingering progenitors could generate grafts with uncontrolled growth when the cells are used for cell therapy and could confound the assay when post-mitotic neurons are needed for disease modeling. Thus, the goal was to optimize the culture to a more mature and post-mitotic population, free of lingering proliferating progenitors. Thus, 3 week-old MGE progenitors were treated with various candidate chemicals/factors for a week and analyzed the proportion of proliferating cells (FIG. 3B). The anti-cancer drug fluorodeoxyuridine (FdU) did not influence the proportion of proliferating MGE progenitors, whereas neuronal culture supplement CultureOne, gamma secretase inhibitor DAPT or CDK4/6 inhibitor PD0332991 significantly reduced the proportion of proliferating progenitors. Furthermore, combined treatment of three working chemicals (termed as CDP) further decreased proliferating cells (FIG. 3C-D and Table S5). As expected, the decrease in the proportion of proliferating cells was accompanied by a decrease in total cell number one week after CDP treatment (FIG. 11). Extending CDP treatment to three weeks further reduced the proportion of proliferating progenitors compared to one week's treatment (FIG. 3E-F and Table S6). The CDP treatment does not affect the phenotypes of treated cell populations (SOX6⁺, GAD⁺, and β-TUBLIN⁺) or cell death even after three weeks' chemical treatment (FIG. 3E-F).

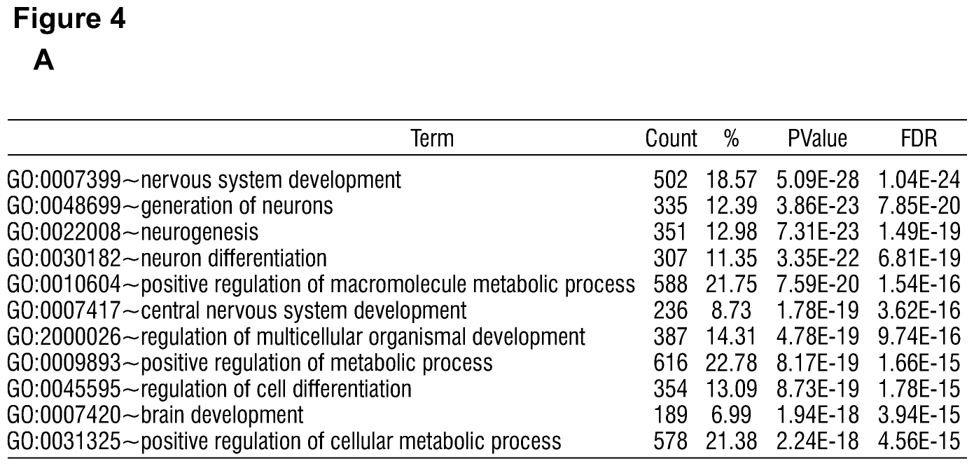

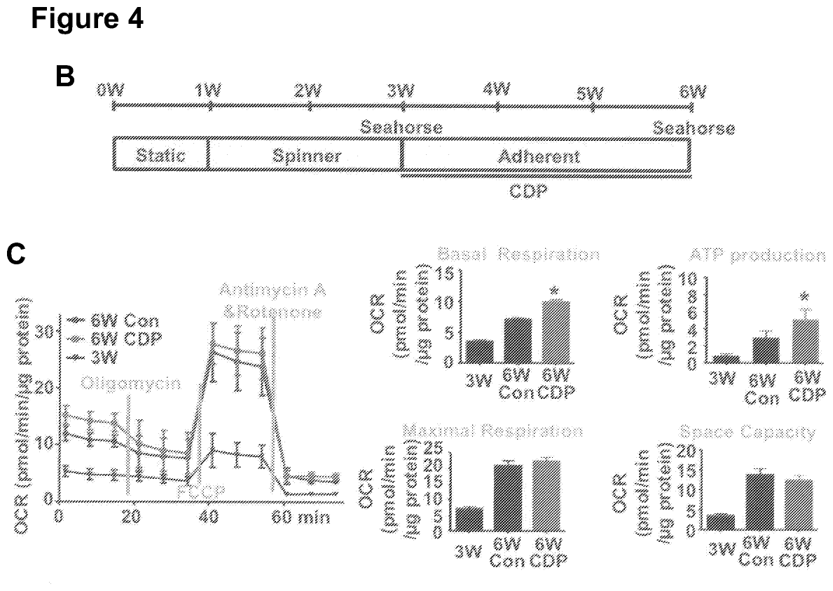

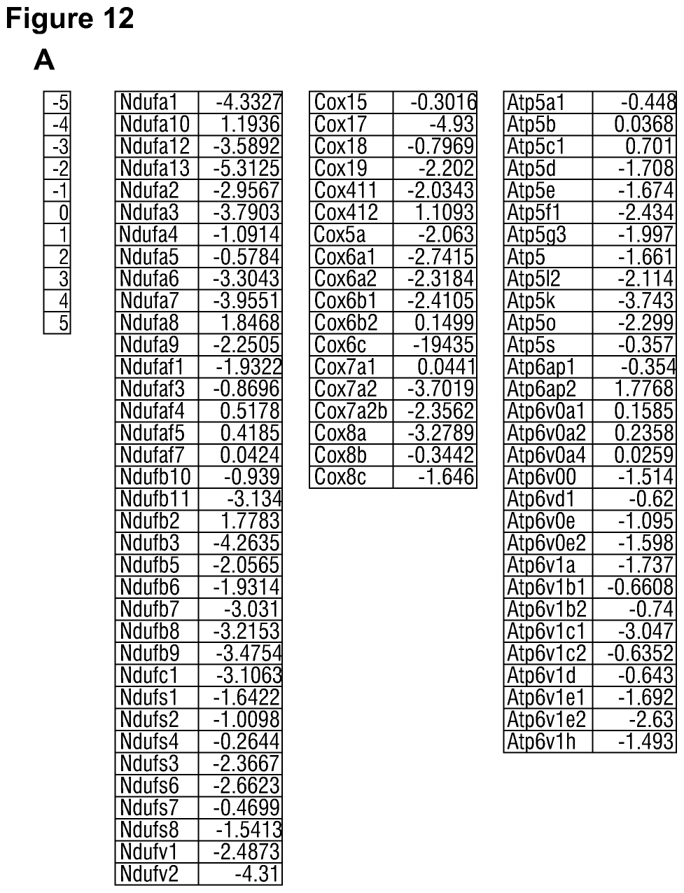

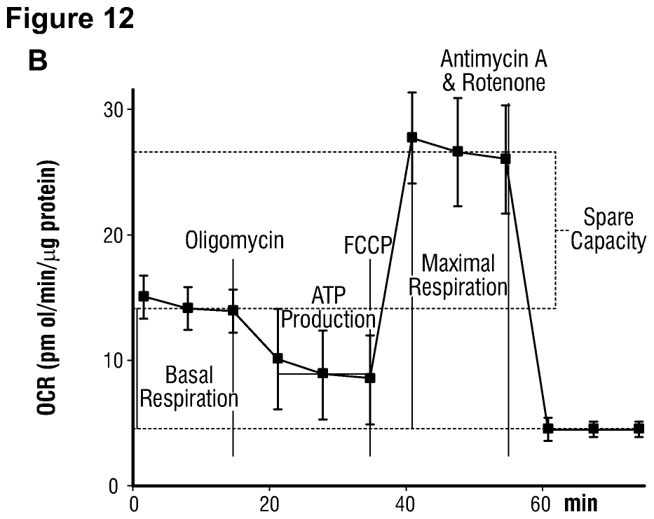

Combined chemical treatment facilitates synchronized maturation of cINs culture. hPSC-derived cINs go through protracted maturation, recapitulating development in vivo (Nicholas et al., Kim et al.). Facilitating maturation to the proper stage for each purpose will be important for efficient use of hPSC-derived cINs for disease modeling and cell therapy. To identify maturation parameters of cINs during development, genes that are differentially expressed during their in vivo development were analyzed, comparing cINs from E13.5 to adult brains (Faux et al., 2010; Okaty et al., 2009). One of the most striking changes during maturation of cINs in mouse brains was the significant upregulation of genes that regulated metabolism (FIG. 3A and FIG. 12A). This developmental change makes sense, considering the high energy demand of mature cINs with fast spiking properties. Thus, metabolic maturation of cINs was analyzed with or without CDP treatment using a Seahorse Analyzer (FIG. 4B and FIG. 12B). CDP-treated cINs showed significant increase in Oxidative Phosphorylation, especially in basal respiration and ATP production (FIG. 4C and Table S7).

During normal development of cINs, they migrate extensively from the MGE all the way to the dorsal telencephalon, where they make local synaptic connections and regulate local circuitry (Wonders and Anderson, 2006). Thus, it was tested whether CDP treatment can facilitate the transformation of MGE progenitors into actively migrating post-mitotic cINs. Thus, 9 week-old cIN organoids were embedded in a Geltrex matrix with or without CDP treatment and their migratory properties were analyzed 7 days after embedding (FIG. 5A-B). There was a significant increase in migratory cINs by CDP treatment compared to untreated cells (FIG. 5B).

As another criterion of maturation, it was analyzed whether CDP treatment affects arborization of cINs. Three week-old cINs were plated on coverslips and labeled only scarcely with a limiting titer of lentivirus that expresses GFP under the Ubiquitin promoter (LV-Ubi-GFP) to facilitate tracing of neurites. Arborization of CDP-treated or untreated cINs was analyzed after 3 weeks' CDP treatment (FIG. 5A). There was a significant increase of arborization with CDP treatment (FIG. 5C and FIG. 13A), shown by the increase in total neurite length (p=0.001, n=12) and total branch numbers (p=0.005, n=12).

Next, the electrophysiological maturation of cINs by whole cell patch clamp after nine weeks' CDP treatment (FIG. 5A) was analyzed. CDP-treated cINs showed more mature membrane properties, including higher resting membrane potential (RMP, P=0.041), lower membrane resistance (Rm, p=0.001), and higher membrane capacitance (Cm, p=0.000). Of the cells recorded, a majority of cINs fired action potentials (APs), with an increased proportion of AP-firing neurons among those treated with CDP. CDP treatment also decreased AP threshold (p=0.046), but there was no significant difference in the parameters of AP firing such as AP half width and AHP (FIG. 13B). 6 week-old cINs were also analyzed with 3 weeks' CDP treatment. The maturation of electrophysiological properties by CDP treatment was significant even at this time point (FIG. 13C), with higher resting membrane potential (RMP, P=0.002), lower membrane resistance (Rm, p=0.025), and higher membrane capacitance (Cm, p=0.0496).

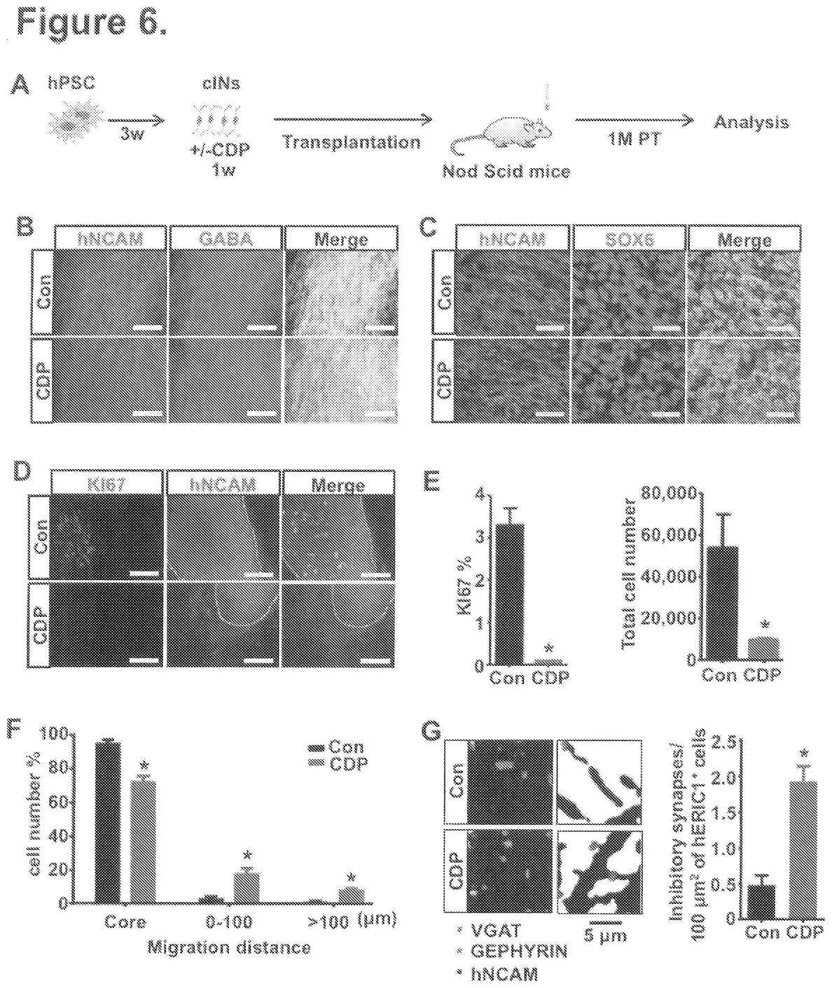

CDP-treated cINs generates safe and well-integrating grafts after transplantation into Nod Scid mice cortex. To further demonstrate that CDP-treated cINs will provide optimal populations for cell transplantation, 4 week-old cINs were grafted with or without a week of CDP treatment to Nod Scid mice cortices and analyzed the grafts one month after transplantation (FIG. 6A). Both untreated and CDP-treated cells generated grafts enriched with the MGE-derived cIN phenotype shown by ubiquitous expression of GABA and SOX6 (FIG. 6B-C). Whereas there are still a number of KI67⁺proliferating cells in untreated grafts, such cells were rarely observed in CDP-treated cell grafts (FIG. 6D-E). Consistent with having more proliferating cells in untreated cell grafts, the total graft cell numbers were significantly higher in untreated cell grafts compared to CDP-treated cell grafts (FIG. 6E). This result gives confidence that CDP-treated cINs will generate safe grafts in the host brain without uncontrolled proliferation.

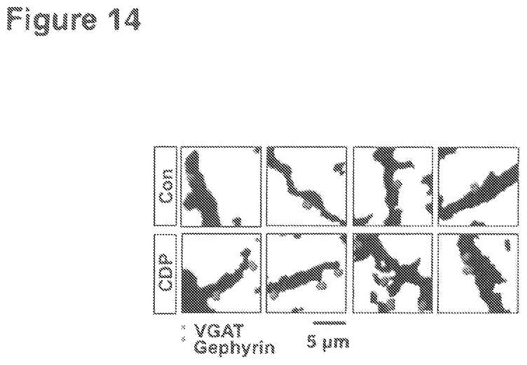

In addition, there are many more migrating cells observed outside the graft core in CDP-treated cell grafts, whereas very few such cells were observed in untreated cell grafts at this time point (FIG. 6D and FIG. 6F). Next, it was tested whether the more mature status of CDP-treated cells would result in more efficient synapse formation of grafted cINs. Thus, inhibitory synapse formation of grafted cINs was analyzed by triple staining the grafted brains with antibodies against human specific NCAM, inhibitory presynaptic marker VGAT, and inhibitory postsynaptic marker Gephyrin. VGAT⁺NCAM⁺ puncta that are juxtaposed with Gephyrin⁺ puncta using IMARIS software were tested. There was a significant increase in inhibitory synapse formation among CDP-treated cINs in the host cortex compared to untreated cINs (FIG. 6G and FIG. 14), suggesting CDP-treated cINs will provide cell populations for optimal integration into host circuitry.

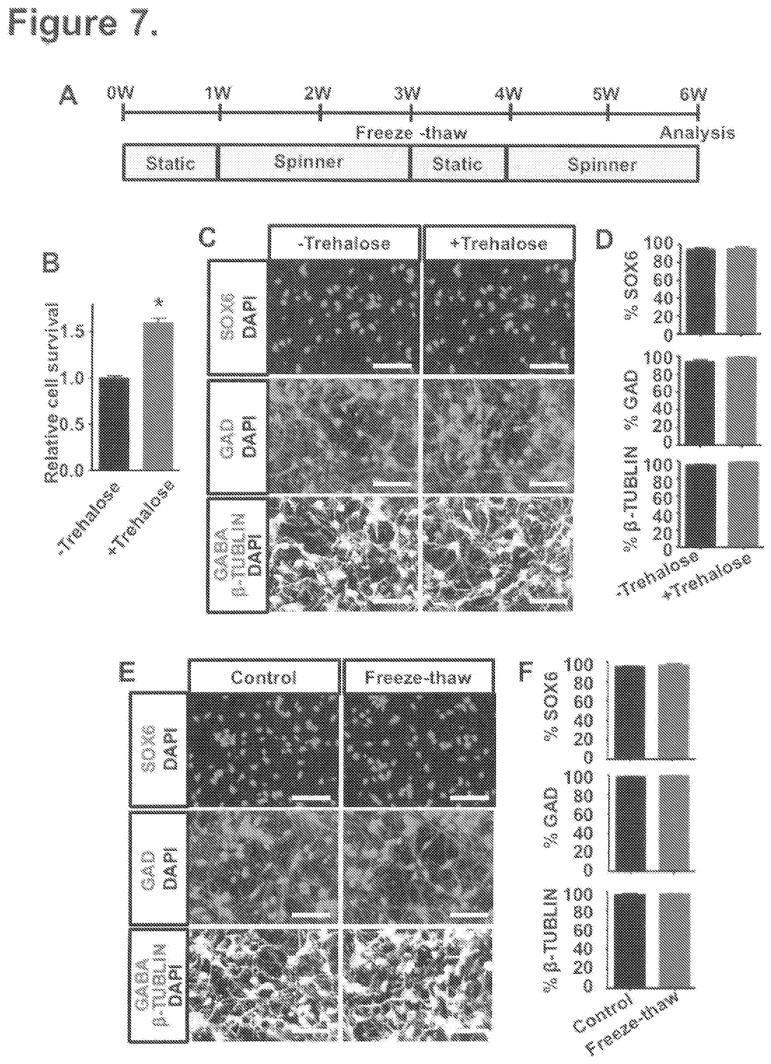

Optimization of cryopreservation of hPSC-derived cINs. Considering the long-term culture required for cIN generation from hPSCs, the ability to freeze down and store intermediate progenies of differentiation will greatly enhance efficiency of their utilization. It was reported that Trehalose could help pluripotent stem cells or other stem cells survive during cryopreservation (Buchanan et al., 2004; Lee et al., 2013; Saxena et al., 2012). Thus, it was tested whether inclusion of Trehalose during cryopreservation of cINs can improve the viability (FIG. 7A). Trehalose indeed significantly increased survival of cryopreserved cINs after freeze-thaw (FIG. 7B), without affecting their phenotypes (FIG. 7C-D). Under the optimized conditions, the majority of cINs maintained viability during cryopreservation under our optimized condition (65.27±8.97% compared to the cINs passaged without cryopreservation). The phenotype of the cells was also well-preserved during freeze-thaw (FIG. 7E-F).

Reliable and efficient generation of human cINs from hPSCs in large scale is critical for further understanding interneuron-related brain disorders and the development of novel therapeutics. The fact that many therapeutics developed in animal models failed in clinical trials in human (Franco and Cedazo-Minguez, 2014; Thomsen et al., 2010) has been raising the need to utilize real human tissues to develop novel therapeutics. In addition, large scale reproducible generation of human cINs from hPSCs will be critical to clinical realization of cell replacement therapy for interneuron-related disorders such as epilepsy, where novel therapeutics are desperately needed, especially for treatment-refractory patients. Previous studies provide a number of methods for generating cINs from hPSCs with varying efficiencies, some using genetic modification (Colasante et al., 2015; Sun et al., 2016; Yang et al., 2017) and others by providing developmentally relevant signaling molecules (Kim et al., 2014; Liu et al., 2013; Maroof et al., 2013; Nicholas et al., 2013). However, large scale generation of cINs that can support efficient drug screening and cell transplantation therapy has not been reported so far. As disclosed herein, different methods of deriving MGE spheres were tested and identified spinner culture support generation of about 20× fold more MGE cells compared to more conventional static culture. The large increase was not due to overgrowth of irrelevant cell types, which was confirmed through immunocytochemical analysis. This method more effectively removes cellular waste from the microenvironment surrounding spheres, and delivers nutrients and oxygens more efficiently by constant stirring. It was still surprising to observe much higher generation of MGE cells compared to shaker culture, which is often used as analogous methods in many organoid cultures (Bagley et al., 2017; Xiang et al., 2017) (Monzel et al., 2017; Ogawa et al., 2018) (Monzel et al., 2017; Ogawa et al., 2018; Qian et al., 2016). One thing noted was that using shaker culture, the spheres tended to adhere each other and formed much larger clusters. This is likely due to centripetal force that tends to keep most of the spheres near the center of the flasks, whereas spheres from spinners form smaller and more homogeneous spheres likely due to the shearing forces generated by stirring motion. Thus, it is possible that the much larger size of shaker-based spheres prevented efficient circulation within spheres (removal of wastes and delivery of nutrients and oxygen), whereas smaller and homogeneous spheres from spinners are better in these terms. Large scale generation accompanied by optimized cryo-preservation using Trehalose provides methods of efficient and versatile use of hPSC-derived cINs for clinical purposes.

To facilitate the maturation of MGE cells, a series of agents were tested that were implicated in cell cycle exit and maturation of neural cell types (Rosen et al., 1989) (Kemp et al., 2016; Schwartz et al., 2011; Telezhkin et al., 2016; Wang et al., 2016). The anti-cancer drug FdU was not effective in reducing immature proliferating cell numbers in MGE cell preparation. However, CultureOne supplement (Life Technologies) that was used to increase neuronal maturation was effective in increasing cell cycle exit of MGE cells. Gamma secretase inhibitor DAPT that was also shown to inhibit notch signaling and thus reduce neuronal progenitor proliferation (Louvi and Artavanis-Tsakonas, 2006; Wang et al., 2016; Yoon and Gaiano, 2005) was also worked well to control cell cycle exit of MGE cells. Cyclin dependent kinase 4/6 (CDK4/6) inhibitor PD0332991 that facilitates cell cycle exit of proliferating neuronal progenitors (Kemp et al., 2016; Schwartz et al., 2011) (Kemp et al., 2016; Schwartz et al., 2011; Telezhkin et al., 2016) was also effective in regulating MGE cell cycle exit. Combination of the last three chemicals was more effective than any single treatment with each chemical alone.

Genetic modification has been used to facilitate MGE phenotype induction or faster maturation (Colasante et al., 2015; Sun et al., 2016; Yang et al., 2017). However, genetic modification methods need to be used with caution for studying diseases of complex genetics such as schizophrenia, where hundreds of common variants (SNPs) work together to cause the phenotype. Thus, a method that can change the SNP landscape would not be desirable. Furthermore, more caution is needed to generate clinical grade cell populations for the purpose of cell therapy, to avoid potential insertional mutation in the grafted cells. Other studies employed feeder co-culture such as astrocytes or glutamatergic neurons for better maturation and maintenance (Colasante et al., 2015; Maroof et al., 2013; Nicholas et al., 2013; Sun et al., 2016). Feeder based cultures also could hinder analysis where a pure population is more desirable (such as transcriptome analysis application) and may not be optimal, especially for clinical application that requires xeno-free preparation of cells. Thus, a chemical combination was used without the use of genetic modification or feeder co-culture to facilitate the maturation of cINs metabolically and functionally (migration, arborization and electrophysiology). Such enhanced maturity will be important for both drug screening purposes to save the time, cost and effort to generate cell populations sufficiently mature for assays. In addition, as shown in this study, this enhanced maturation will also be critical to avoiding uncontrolled growth of proliferating cells in grafts and better integration of grafted cells into the host brain with superior migration, arborization and synaptic connection.

It is well-known that human cINs derived from hPSCs are undergoing protracted maturation comparable to their in vivo developmental time line. Continued maturation has been observed during the culture of hPSC-derived cINs just as during normal development (Kim et al., 2014; Nicholas et al., 2013), and our protocol with chemical treatment significantly facilitates this process. Some applications of hPSC-derived cINs, such as modeling the disease phenotype in adult brains, will require attainment of fully mature phenotype. However, for the purpose of cell therapy, this type of developing migratory cINs (SOX6⁺ KI67⁻) will be more beneficial than completely mature neurons in that 1) completely mature neurons with elaborate neurites will be more vulnerable to the passaging and transplantation procedure than developing cINs and 2) completely mature neurons lacks migratory properties for optimal integration into host circuitry unlike these developing early postmitotic cINs. They will also be better suited for cell therapy compared to more immature progenitors in that 1) they contain all the machinery to readily integrate into host circuitry and regulate host circuitry and 2) they are post-mitotic without any more proliferating cells without the chance of uncontrolled growth of grafted cells. As disclosed herein, efficient large-scale generation of hPSC-derived human cINs with the proper developmental stage optimal for cell transplantation is shown, and provides critical tools for efficient use of hPSC-derived cINs for cell therapy.

TABLE S1
Chemical list used in the experiments. Related to Method section.

| Chemicals | | | | | |
|---|---|---|---|---|---|
| Full name | Abbreviations | Vendor | Cat No | Concentration | |
| 5-fluoro-2′-deoxyuridine | FdU | Sigma-Aldrich | 856657 | 10 | μM |
| Ascorbic acid | AA | Sigma-Aldrich | 1043003 | 200 | mM |
| B27 ™ Supplement (50X), serum free | B27 | Thermo Fisher | 17504044 | 1% | |
| β-mercaptoethanol | — | Thermo Fisher | 21985023 | 10 | μM |
| Boc-Asp (OMe) fluoromethyl ketone | BAF | Sigma-Aldrich | B2682 | 20 | μM |
| Brain-derived neurotrophic factor | BDNF | ProSpec Bio | CYT-207 | 5 | ng/ml |
| CultureOne Supplement | C | Thermo Fisher | A3320201 | 1% | |
| DAPT | D | Sigma-Aldrich | D5942 | 2.51 | μM |
| Dulbecco's Modified Eagle Medium, high glucose | DMEM | Thermo Fisher | 11965118 | — | |
| Dulbecco's Modified Eagle Medium/ | DMEM/F12 | Thermo Fisher | 11330032 | — | |
| Nutrient Mixture F-12 | | | | | |
| Essential 8 Flex Medium Kit | E8 | Thermo Fisher | A2858501 | — | |
| FGF8 | — | Peprotech | CYT-839 | 50 | ng/ml |
| Fibronectin | FN | Thermo Fisher | 33016015 | 10 | μg/mL |
| Geltrex LDEV-free hESC Qualified, Reduced | Geltrex | Thermo Fisher | A1413302 | 150 | μg/ml |
| Growth Factor Basement Membrane Matrix | | | | | |
| Glial cell-derived neurotrophic factor | GDNF | ProSpec Bio | CYT-305 | 5 | ng/ml |
| Glucose | — | Thermo Fisher | A24940-01 | 10 | mM |
| Hank's Balanced Salt Solution | HBSS | Thermo Fisher | 14025092 | — | |
| HyClone Fetal Bovine Serum (U.S.), Defined | FBS | Hyclone | SH3007003 | 10% | |
| IWP2 | W | Selleck Chem | s7085 | 5 | μM |
| KnockOut Serum Replacement | KSR | Thermo Fisher | 10828028 | 15% | |
| LDN193189 | L | Selleck Chem | S2618 | 0.11 | μM |
| L-glutamine | — | Thermo Fisher | 2503081 | 2 | mM |
| M-MLV reverse transcriptase | — | Thermo Fisher | 28025021 | 200 | U/μl |
| N-2 Supplement | N2 | Thermo Fisher | 17502048 | 0.5% | |
| Optimum cutting temperature Compound | O.C.T. | Thermo Fisher | 4585 | — | |
| Paraformaldehyde | PFA | Sigma-Aldrich | 252549 | 4% | |
| PD0332991 | P | Sigma-Aldrich | PZ0199 | 2 | μM |
| Pentobarbital | — | Sigma-Aldrich | 1507002 | 150 | mg/kg |
| Phosphate buffered saline | PBS | Sigma-Aldrich | P4417 | 1 tablet/200 ml | |
| Poly-L-ornithine | PLO | Sigma-Aldrich | P4957 | 15 | μg/mL |
| SB431452 | S | Tocris Bioscience | 1614 | 10 | μM |
| Smoothened Agonist (SAG) | Sg | Selleck Chem | S7779 | 0.1 | μM |
| Sodium pyruvate | — | Thermo Fisher | 11360-070 | 5 | mM |
| SsoAdvanced Universal SYBR Green Supermix | — | Bio-Rad | 172-5272 | — | |
| Sucrose | — | Thermo Fisher | FLS5-500 | 30% | |
| Thehalose | — | Sigma-Aldrich | T0167 | 10 | mM |
| TRIzol | — | Thermo Fisher | 15596026 | — | |
| Trypsin | — | Thermo Fisher | 25300054 | 0.05% | |
| Turbo DNase | — | Thermo Fisher | AM2238 | 2 | U/ml |
| ROCK inhibitor, Y27632 | Y | Apex Bio | A3008 | 10 | μM |

TABLE S2
Antibody list used in the experiments. Related to Methods section.

| Antibody | Species | Source | Cat No. | Dilution |
|---|---|---|---|---|
| β-TUBLIN | Mouse | BD Biosciences | BD560381 | 1:1000 |
| Cleaved CASPASE 3 | Rabbit | Cell signaling | 9661 | 1:1000 |
| (Asp175) | | | | |
| DCX | Guinea pig | Thermo Fisher | AB2753 | 1:1000 |
| DLX2 | Rabbit | Dr. Morozov | — | 1:5000 |
| | | (Morozov et al., 2009) | | |
| GABA | Rabbit | Immunostar | 20094 | 1:1000 |
| GAD 67 (N-19) | Goat | Santa Cruz | SC7512 | 1:1000 |
| KI67 | Mouse | Abcam | Ab16667 | 1:1000 |
| NESTIN | Mouse | Santa Cruz | SC-33677 | 1:1000 |
| NKX2.1 (TTF1, H-190) | Rabbit | Santa Cruz | SC-13040X | 1:1000 |
| SOX2 (E-4) | Mouse | Santa Cruz | SC-365823 | 1:1000 |
| SOX6 | Rabbit | Thermo Fisher | AB5805 | 1:1000 |

TABLE S3
Spinner culture generated significantly higher yield of H9 MGE spheres with
more homogeneous sizes. Related to FIG. 1B.

| | | | | | One-way | Tukey post-hoc analysis | | |
|---|---|---|---|---|---|---|---|---|
| | | | | | ANOVA | Static vs. | Static vs. | Shaker vs. |
| Group | | Average | SEM | N | p value | Shaker | Spinner | Spinner |
| Static | Relative fold | 1.000 | 0.234 | 6 | <0.001 | 0.909 | <0.001 | <0.001 |
| Shaker | change of cell | 1.954 | 0.421 | 6 | | | | |
| Spinner | number | 20.406 | 2.754 | 6 | | | | |
| Static | Sphere | 400.000 | 54.281 | 8 | <0.001 | 0.003 | 0.101 | <0.001 |
| Shaker | sizes | 800.954 | 123.443 | 7 | | | | |
| Spinner | (□m) | 200.406 | 23.241 | 9 | | | | |

TABLE S4
qPCR analysis of H9 cIN organoids at different time points. Related to FIG. 2D.

| | Relative gene expression | | | One-way | Tukey post-hoc analysis | | |
|---|---|---|---|---|---|---|---|
| | (Average ± SEM, N) | | | ANOVA | 0W vs. | 0W vs. | 4W vs. |
| Group | 0W | 4W | 40W | p value | 4W | 40W | 4W |
| K167 | 1.000 ± 0.244, 3 | 2.011 ± 0.334, 3 | 0.4960 ± 0.290, 3 | 0.027 | 0.167 | 0.595 | 0.011 |
| GAD | 1.000 ± 1.079, 3 | 659.821 ± 304.212, 3 | 8.005e3 ± 2.236e3, 3 | 0.009 | 0.933 | 0.012 | 0.017 |
| SOX6 | 1.000 ± 0.1000, 3 | 1.068e4 ± 4.991e3, 3 | 3.668e5 ± 8.668e4, 3 | 0.003 | 0.988 | 0.005 | 0.006 |
| DLX2 | 1.000 ± 1.710, 3 | 700.743 ± 323.421, 3 | 2.468e4 ± 3.118e3, 3 | <0.001 | 0.960 | <0.001 | <0.001 |
| LHX6 | 1.000 ± 1.753, 3 | 1.890 ± 0.871, 3 | 4.560e3 ± 1.633e3, 3 | 0.022 | 0.999 | 0.033 | 0.033 |
| SST | 1.000 ± 2.841, 3 | 271.823 ± 125.573, 3 | 9.450e4 ± 1.605e4, 3 | 0.001 | 0.999 | <0.001 | <0.001 |

TABLE S5
Combined chemical treatment significantly decreased proportion of proliferating cells. Related to FIG. 3D.

| | Con | FdU | Culture One | DAPT | PD0332991 | CDP |
|---|---|---|---|---|---|---|
| Percentage of proliferating cells | 30.721 ± 2.157, 4 | 31.719 ± 1.486, 4 | 8.171 ± 1.145, 4 | 8.835 ± 1.618, 4 | 5.160 ± 1.042, 4 | 2.493 ± 0.397 ,4 |
| (Average ± SEM, N) | | | | | | |

| One-way ANOVA p value | | <0.001 |
|---|---|---|
| Tukey | FdU vs. Con | 0.763 |
| post-hoc | CultureOne vs. Con | <0.001 |
| analysis | DAPT vs. Con | <0.001 |
| | PD0332991 vs. Con | <0.001 |
| | CDP vs. Con | <0.001 |
| | CultureOne vs. FdU | <0.001 |
| | DAPT vs. FdU | <0.001 |
| | PD0332991 vs. FdU | <0.001 |
| | DAPT vs. CultureOne | 0.991 |
| | PD0332991 vs. CultureOne | 0.843 |
| | CDP vs. CultureOne | 0.006 |
| | PD0332991 vs .DAPT | 0.990 |
| | CDP vs. DAPT | 0.015 |
| | CDP vs. PD0332991 | 0.040 |

TABLE S6
Combined chemical treatment significantly decreased proportion
of H9-generated proliferating cells. Related to FIG. 3F.

| | One-way | Tukey post-hoc analysis | | |
|---|---|---|---|---|
| Treatment | ANOVA | Con vs. | Con vs. | 1 W vs. |
| Group | p value | 1 W | 3 W | 3 W |
| Control | <0.001 | 0.001 | <0.001 | 0.098 |
| 1 week | | | | |
| 3 weeks | | | | |

TABLE S7
CDP-treatment significantly increased Oxidative Phosphorylation of cINs. Related to FIG. 4C.

| | Fold change of gene expression | | | One-way | Tukey post-hoc analysis | | |
|---|---|---|---|---|---|---|---|
| | (Average ± SEM, N) | | | ANOVA | 3W vs. | 3W vs. | 6W Con vs. |
| Group | 3W | 6W Con | 6W CDP | p value | 6W Con | 6W CDP | 6W CDP |
| Basal | 3.537 ± 0.249, 10 | 7.207 ± 0.196, 10 | 9.682 ± 0.384, 10 | <0.001 | <0.001 | <0.001 | <0.001 |
| Respiration | | | | | | | |
| ATP | 0.874 ± 0.155, 10 | 3.029 ± 0.746, 10 | 5.177 ± 1.155, 10 | 0.002 | 0.019 | 0.009 | 0.023 |
| production | | | | | | | |
| Maximum | 7.038 ± 0.708, 10 | 20.630 ± 1.439, 10 | 22.307 ± 1.179, 10 | <0.001 | <0.001 | <0.001 | 0.481 |
| Respiration | | | | | | | |
| Space | 3.500 ± 0.467, 10 | 13.423 ± 1.448, 10 | 12.446 ± 1.277, 10 | <0.001 | <0.001 | <0.001 | 0.745 |
| Capacity | | | | | | | |

### REFERENCES

Each of the reference cited within the specification and those listed below are hereby incorporated by reference in their entirety.

Ahn, S., Kim, T. G., Kim, K. S., and Chung, S. (2016). Differentiation of human pluripotent stem cells into Medial Ganglionic Eminence vs. Caudal Ganglionic Eminence cells. Methods 101, 103-112.

Amariglio, N., Hirshberg, A., Scheithauer, B. W., Cohen, Y., Loewenthal, R., Trakhtenbrot, L., Paz, N., Koren-Michowitz, M., Waldman, D., Leider-Trejo, L., et al. (2009). Donor-derived brain tumor following neural stem cell transplantation in an ataxia telangiectasia patient. PLoS medicine 6, e1000029.

Bagley, J. A., Reumann, D., Bian, S., Levi-Strauss, J., and Knoblich, J. A. (2017). Fused cerebral organoids model interactions between brain regions. Nature methods 14, 743-751.

Bandler, R. C., Mayer, C., and Fishell, G. (2017). Cortical interneuron specification: the juncture of genes, time and geometry. Current opinion in neurobiology 42, 17-24.

Berkowitz, A. L., Miller, M. B., Mir, S. A., Cagney, D., Chavakula, V., Guleria, I., Aizer, A., Ligon, K. L., and Chi, J. H. (2016). Glioproliferative Lesion of the Spinal Cord as a Complication of “Stem-Cell Tourism”. The New England journal of medicine 375, 196-198.

Braz, J. M., Sharif-Naeini, R., Vogt, D., Kriegstein, A., Alvarez-Buylla, A., Rubenstein, J. L., and Basbaum, A. I. (2012). Forebrain GABAergic neuron precursors integrate into adult spinal cord and reduce injury-induced neuropathic pain. Neuron 74, 663-675.

Buchanan, S. S., Gross, S. A., Acker, J. P., Toner, M., Carpenter, J. F., and Pyatt, D. W. (2004). Cryopreservation of stem cells using trehalose: evaluation of the method using a human hematopoietic cell line. Stem cells and development 13, 295-305.

Carvalho, B. S., and Irizarry, R. A. (2010). A framework for oligonucleotide microarray preprocessing. Bioinformatics 26, 2363-2367.

Colasante, G., Lignani, G., Rubio, A., Medrihan, L., Yekhlef, L., Sessa, A., Massimino, L., Giannelli, S. G., Sacchetti, S., Caiazzo, M., et al. (2015). Rapid Conversion of Fibroblasts into Functional Forebrain GABAergic Interneurons by Direct Genetic Reprogramming. Cell stem cell 17, 719-734.

Cunningham, M., Cho, J. H., Leung, A., Savvidis, G., Ahn, S., Moon, M., Lee, P. K., Han, J. J., Azimi, N., Kim, K. S., et al. (2014). hPSC-derived maturing GABAergic interneurons ameliorate seizures and abnormal behavior in epileptic mice. Cell stem cell 15, 559-573.

Donegan, J. J., Tyson, J. A., Branch, S. Y., Beckstead, M. J., Anderson, S. A., and Lodge, D. J. (2017). Stem cell-derived interneuron transplants as a treatment for schizophrenia: preclinical validation in a rodent model. Molecular psychiatry 22, 1492-1501.

Faux, C., Rakic, S., Andrews, W., Yanagawa, Y., Obata, K., and Parnavelas, J. G. (2010). Differential gene expression in migrating cortical interneurons during mouse forebrain development. The Journal of comparative neurology 518, 1232-1248.

Franco, R., and Cedazo-Minguez, A. (2014). Successful therapies for Alzheimers disease: why so many in animal models and none in humans? Frontiers in pharmacology 5, 146.

Gautier, L., Cope, L., Bolstad, B. M., and Irizarry, R. A. (2004). affy—analysis of Affymetrix GeneChip data at the probe level. Bioinformatics 20, 307-315.

Goldman, S. A. (2016). Stem and Progenitor Cell-Based Therapy of the Central Nervous System: Hopes, Hype, and Wishful Thinking. Cell stem cell 18, 174-188.

Hoffman, G. E., Hartley, B. J., Flaherty, E., Ladran, I., Gochman, P., Ruderfer, D. M., Stahl, E. A., Rapoport, J., Sklar, P., and Brennand, K. J. (2017). Transcriptional signatures of schizophrenia in hiPSC-derived NPCs and neurons are concordant with post-mortem adult brains. Nature communications 8, 2225.

Hong, S., Hwang, D. Y., Yoon, S., Isacson, O., Ramezani, A., Hawley, R. G., and Kim, K. S. (2007). Functional analysis of various promoters in lentiviral vectors at different stages of in vitro differentiation of mouse embryonic stem cells. Mol Ther 15, 1630-1639.

Irizarry, R. A., Hobbs, B., Collin, F., Beazer-Barclay, Y. D., Antonellis, K. J., Scherf, U., and Speed, T. P. (2003). Exploration, normalization, and summaries of high density oligonucleotide array probe level data. Biostatistics 4, 249-264.

Kauffmann, A., Gentleman, R., and Huber, W. (2009). arrayQualityMetrics—a bioconductor package for quality assessment of microarray data. Bioinformatics 25, 415-416.

Kemp, P. J., Rushton, D. J., Yarova, P. L., Schnell, C., Geater, C., Hancock, J. M., Wieland, A., Hughes, A., Badder, L., Cope, E., et al. (2016). Improving and accelerating the differentiation and functional maturation of human stem cell-derived neurons: role of extracellular calcium and GABA. The Journal of physiology 594, 6583-6594.

Kikuchi, T., Morizane, A., Doi, D., Magotani, H., Onoe, H., Hayashi, T., Mizuma, H., Takara, S., Takahashi, R., Inoue, H., et al. (2017). Human iPS cell-derived dopaminergic neurons function in a primate Parkinsons disease model. Nature 548, 592-596.

Kim, T. G., Yao, R., Monnell, T., Cho, J. H., Vasudevan, A., Koh, A., Peeyush, K. T., Moon, M., Datta, D., Bolshakov, V. Y., et al. (2014). Efficient specification of interneurons from human pluripotent stem cells by dorsoventral and rostrocaudal modulation. Stem cells 32, 1789-1804.

Lee, Y. A., Kim, Y. H., Kim, B. J., Kim, B. G., Kim, K. J., Auh, J. H., Schmidt, J. A., and Ryu, B. Y. (2013). Cryopreservation in trehalose preserves functional capacity of murine spermatogonial stem cells. PloS one 8, e54889.

Liu, Y., Weick, J. P., Liu, H., Krencik, R., Zhang, X., Ma, L., Zhou, G. M., Ayala, M., and Zhang, S. C. (2013). Medial ganglionic eminence-like cells derived from human embryonic stem cells correct learning and memory deficits. Nature biotechnology 31, 440-447.

Louvi, A., and Artavanis-Tsakonas, S. (2006). Notch signalling in vertebrate neural development. Nature reviews Neuroscience 7, 93-102.

Marin, O. (2012). Interneuron dysfunction in psychiatric disorders. Nature reviews Neuroscience 13, 107-120.

Maroof, A. M., Keros, S., Tyson, J. A., Ying, S. W., Ganat, Y. M., Merkle, F. T., Liu, B., Goulburn, A., Stanley, E. G., Elefanty, A. G., et al. (2013). Directed differentiation and functional maturation of cortical interneurons from human embryonic stem cells. Cell stem cell 12, 559-572.

Martinez-Cerdeno, V., Noctor, S. C., Espinosa, A., Ariza, J., Parker, P., Orasji, S., Daadi, M. M., Bankiewicz, K., Alvarez-Buylla, A., and Kriegstein, A. R. (2010). Embryonic MGE precursor cells grafted into adult rat striatum integrate and ameliorate motor symptoms in 6-OHDA-lesioned rats. Cell stem cell 6, 238-250.

Monzel, A. S., Smits, L. M., Hemmer, K., Hachi, S., Moreno, E. L., van Wuellen, T., Jarazo, J., Walter, J., Bruggemann, I., Boussaad, I., et al. (2017). Derivation of Human Midbrain-Specific Organoids from Neuroepithelial Stem Cells. Stem cell reports 8, 1144-1154.

Nicholas, C. R., Chen, J., Tang, Y., Southwell, D. G., Chalmers, N., Vogt, D., Arnold, C. M., Chen, Y. J., Stanley, E. G., Elefanty, A. G., et al. (2013). Functional maturation of hPSC-derived forebrain interneurons requires an extended timeline and mimics human neural development. Cell stem cell 12, 573-586.

Ogawa, J., Pao, G. M., Shokhirev, M. N., and Verma, I. M. (2018). Glioblastoma Model Using Human Cerebral Organoids. Cell reports 23, 1220-1229.

Okaty, B. W., Miller, M. N., Sugino, K., Hempel, C. M., and Nelson, S. B. (2009). Transcriptional and electrophysiological maturation of neocortical fast-spiking GABAergic interneurons. The Journal of neuroscience: the official journal of the Society for Neuroscience 29, 7040-7052.

Qian, X., Nguyen, H. N., Song, M. M., Hadiono, C., Ogden, S. C., Hammack, C., Yao, B., Hamersky, G. R., Jacob, F., Zhong, C., et al. (2016). Brain-Region-Specific Organoids Using Mini-bioreactors for Modeling ZIKV Exposure. Cell 165, 1238-1254.

Rhee, Y. H., Ko, J. Y., Chang, M. Y., Yi, S. H., Kim, D., Kim, C. H., Shim, J. W., Jo, A. Y., Kim, B. W., Lee, H., et al. (2011). Protein-based human iPS cells efficiently generate functional dopamine neurons and can treat a rat model of Parkinson disease. The Journal of clinical investigation 121, 2326-2335.

Rosen, C. L., Bunge, R. P., Ard, M. D., and Wood, P. M. (1989). Type 1 astrocytes inhibit myelination by adult rat oligodendrocytes in vitro. The Journal of neuroscience: the official journal of the Society for Neuroscience 9, 3371-3379.

Saxena, A., Wagatsuma, A., Noro, Y., Kuji, T., Asaka-Oba, A., Watahiki, A., Gurnot, C., Fagiolini, M., Hensch, T. K., and Carninci, P. (2012). Trehalose-enhanced isolation of neuronal sub-types from adult mouse brain. BioTechniques 52, 381-385.

Schwartz, G. K., LoRusso, P. M., Dickson, M. A., Randolph, S. S., Shaik, M. N., Wilner, K. D., Courtney, R., and ODwyer, P. J. (2011). Phase I study of PD 0332991, a cyclin -dependent kinase inhibitor, administered in 3-week cycles (Schedule 2/1). British journal of cancer 104, 1862-1868.

Southwell, D. G., Nicholas, C. R., Basbaum, A. I., Stryker, M. P., Kriegstein, A. R., Rubenstein, J. L., and Alvarez-Buylla, A. (2014). Interneurons from embryonic development to cell-based therapy. Science 344, 1240622.

Sun, A. X., Yuan, Q., Tan, S., Xiao, Y., Wang, D., Khoo, A. T., Sani, L., Tran, H. D., Kim, P., Chiew, Y. S., et al. (2016). Direct Induction and Functional Maturation of Forebrain GABAergic Neurons from Human Pluripotent Stem Cells. Cell reports 16, 1942-1953.

Tabar, V., and Studer, L. (2014). Pluripotent stem cells in regenerative medicine: challenges and recent progress. Nature reviews Genetics 15, 82-92.

Takahashi, K., and Yamanaka, S. (2006). Induction of pluripotent stem cells from mouse embryonic and adult fibroblast cultures by defined factors. Cell 126, 663-676.

Telezhkin, V., Schnell, C., Yarova, P., Yung, S., Cope, E., Hughes, A., Thompson, B. A., Sanders, P., Geater, C., Hancock, J. M., et al. (2016). Forced cell cycle exit and modulation of GABAA, CREB, and GSK3beta signaling promote functional maturation of induced pluripotent stem cell-derived neurons. American journal of physiology Cell physiology 310, C520-541.

Thomsen, M. S., Hansen, H. H., Timmerman, D. B., and Mikkelsen, J. D. (2010). Cognitive improvement by activation of alpha7 nicotinic acetylcholine receptors: from animal models to human pathophysiology. Current pharmaceutical design 16, 323-343.

Wang, J., Ye, Z., Zheng, S., Chen, L., Wan, Y., Deng, Y., and Yang, R. (2016). Lingo-1 shRNA and Notch signaling inhibitor DAPT promote differentiation of neural stem/progenitor cells into neurons. Brain research 1634, 34-44.

Wonders, C., and Anderson, S. A. (2005). Cortical interneurons and their origins. The Neuroscientist: a review journal bringing neurobiology, neurology and psychiatry 11, 199-205.

Wonders, C. P., and Anderson, S. A. (2006). The origin and specification of cortical interneurons. Nature reviews Neuroscience 7, 687-696.

Xiang, Y., Tanaka, Y., Patterson, B., Kang, Y. J., Govindaiah, G., Roselaar, N., Cakir, B., Kim, K. Y., Lombroso, A. P., Hwang, S. M., et al. (2017). Fusion of Regionally Specified hPSC-Derived Organoids Models Human Brain Development and Interneuron Migration. Cell stem cell 21, 383-398 e387.

Yang, N., Chanda, S., Marro, S., Ng, Y. H., Janas, J. A., Haag, D., Ang, C. E., Tang, Y., Flores, Q., Mall, M., et al. (2017). Generation of pure GABAergic neurons by transcription factor programming. Nature methods 14, 621-628.

Yoon, K., and Gaiano, N. (2005). Notch signaling in the mammalian central nervous system: insights from mouse mutants. Nat Neurosci 8, 709-715.

Zhao, C., Wang, Q., and Temple, S. (2017). Stem cell therapies for retinal diseases: recapitulating development to replace degenerated cells. Development 144, 1368-1381.

Zhu, Q., Naegele, J. R., and Chung, S. (2018). Cortical GABAergic Interneuron/Progenitor Transplantation as a Novel Therapy for Intractable Epilepsy. Frontiers in cellular neuroscience 12, 167.

What is claimed:

1. A method of generating MGE progenitors from pluripotent stem cells said method comprising the step of establishing sphere cultures from pluripotent stem cells under spinner culture conditions.

2. A method of generating a synchronized culture of cINs, derived from pluripotent stem cells, comprising the step of culturing of said cells in the presence of one or more of the following components: CultureOne, gamma secretase inhibitor DAPT and CDK4/6 inhibitor PD0332991.

3. The method of claim 2, wherein the concentration of Culture One is in the range of 1% -10%.

4. The method of claim 2, wherein the concentration of DAPT is in a range of about 3-10 μM.

5. The method of claim 2, wherein the concentration of PD0332991 is in the range of about 1-8 μM.

6. The method of claim 2, wherein the components of CDP are 1% CultureOne, 10 μM DAPT and 2 μM PD0332991

7. The method of claim 2, wherein the step of culturing is done in the presence of Trehalose.

8. The method of claim 7, wherein the concentration of Trehalose is between about 0.1-0.4M.

9. The method of claim 8, wherein the concentration of Trehalose is about 0.1 M.

10. A method of cryopreserving synchronized cultures of cINs wherein said interneurons are cryopreserved in the presence of Trehalose.

11. A population of synchronized cINs derived using the method of claim 2.

12. A method of treating a host in need of said treatment comprising transplantation of the population of synchronized cINs of claim 11 into the host.

13. The method of claim 12, wherein the host is in need of treatment of a neurological disorder.

14. The method of claim 12, wherein the cINS are autologous cells.

15. The method of claim 12, wherein the cINs are allogenic.

16. The method of claim 2, wherein said cINs are characterized by the expression of one or more of the following markers, SOX6+, GAD+, B-tubulin+, NKX2.1, SST and MEF2C.
